(19) United States
(12) Patent Application Publication
(10) Pub. No.: US 2006/0258668 A1
(43) Pub. Date: Nov. 16, 2006

(54) Use of phosphatase inhibitors as adjunct therapy for psychiatric disorders

(76) Inventor: Edward M. Scolnick, Wynnewood, PA (US)

Correspondence Address: MERCK AND CO., INC, P O BOX 2000, RAHWAY, NJ, 07065-0907, US

(21) Appl. No.: 10/557,648

(22) PCT Filed: May 25, 2004
(86) PCT No.: PCT/US04/16492

§ 371 Date: Nov. 18, 2005

Related U.S. Application Data
(60) Provisional application No. 60/474,168, filed on May 29, 2003.

(51) Int. Cl. A61K 31/519; A61K 31/4745; A61K 31/4709; A61K 31/4439; A61K 31/47

(52) U.S. Cl. 514/252.16; 514/311; 514/314; 514/340; 514/300

(57) ABSTRACT

The use of a phosphatase inhibitor in conjunction with psychotherapy provides enhanced therapeutic results in the treatment of psychiatric disorders including, for example, specific phobias, panic disorders, anxiety disorders including posttraumatic stress disorders, and obsessive-compulsive disorder.

### BACKGROUND OF THE INVENTION

This Patent Application relates to the use of phosphatase inhibitors as an adjunct in the treatment of psychiatric disorders. In particular, this Patent Application relates to the use of Calcineurin (CN) inhibitors and protein phosphatase 1 (PP1) inhibitors as adjunct therapy in behavior psychotherapy, cognitive psychotherapy, psychodynamically oriented psychotherapy and in the treatment of posttraumatic stress disorder.

The Classical fear conditioning occurs when an affectively neutral stimulus is paired with a noxious aversive stimulus (unconditioned stimulus (US)) such as footshock.

Afterward, the previously neutral stimulus (i.e., now the conditioned stimulus (CS)) is able to elicit a variety of autonomic, hormonal, and skeletal responses that accompany the conscious experience of fear in humans and which are used to operationally define fear in laboratory animals. The fear-eliciting properties of the CS can be extinguished by repeatedly presenting the CS in the absence of the US.

A reduced ability to extinguish intense fear memories is a significant clinical problem for a wide range of psychiatric disorders including specific phobias, panic disorder, and posttraumatic stress disorder. Because treatment of these disorders often relies upon the progressive extinction of fear memories, pharmacological enhancement of extinction could be of considerable clinical benefit in these conditions.

Recently, Lin, et. al., published studies which are said to “. . . show that activation of calcineurnin contributes to the extinction.” See J. Neuoroscience, vol. 23, 1574-1579, 2003.

There is evidence that hippocampal-dependent synaptic plasticity and memory storage depend (at least in part) on the balance between phosphorylation and dephosphorylation mediated by cAMP-dependent protein kinase (PKA) and the phosphatase calicineurin (CN). PKA, on one hand, appear to be important for the initiation of synaptic plasticity and for learning and memory. PKA also appears to be required for persistent long term potentiation (LPT), important for synaptic strengthening in learning and memory. CN, on the other hand, inhibits synaptic plasticity and memory storage. Similarly, PP1 appears to promote forgetting. Recent studies by Malleret, et. al., Cell, vol. 104, 675-686, 2001, indicate that the balance can be shifted in favor of learning and memory storage by inhibiting CN.

Thus, the theory underlying Lin, et. al, appears to be that extinction of fear memory is promoted by enhancing the subjects capacity to either forget the original traumatic event or forget the linkage between original traumatic event and the stimulus that reminds the subject of that event.

To the contrary, we have concluded that enhancement of new memory acquisition and consolidation rather than enhancement in the ability forget undesired memories, lies at the core of the effective extinction of fear. Accordingly, we have concluded that inhibition (rather than enhancement) of CN or PP1, in conjunction with appropriate therapy is effective in the extinction of fear.

Hormones are compounds that variously affect cellular activity. In many respects, hormones act as messengers to trigger specific cellular responses and activities. Many effects produced by hormones, however, are not caused by the singular effect of just the hormone.

Instead, the hormone first binds to a receptor, thereby triggering the release of a second compound that goes on to affect the cellular activity. In this scenario, the hormone is known as the first messenger while the second compound is called the second messenger. Cyclic adenosine monophosphate (adenosine 3′,5′-cyclic monophosphate, “cAMP” or “cyclic AMP”) is known as a second messenger for hormones including epinephrine, glucagon, calcitonin, corticotrophin, lipotropin, luteinizing hormone, norepinephrine, parathyroid hormone, thyroid-stimulating hormone, and vasopressin. Thus, cAMP mediates cellular responses to hormones. Cyclic AMP also mediates cellular responses to various neurotransmitters.

Phosphodiesterases (“PDE”) are a family of enzymes that metabolize 3′,5′-cyclic nucleotides to 5′-nucleoside monophosphates, thereby terminating cAMP second messenger activity. A particular phosphodiesterase, phosphodiesterase-4 (“PDE4”, also known as “PDE-IV”), which is a high affinity, cAMP specific, type IV PDE, has generated interest as potential targets for the development of novel pharmaceuticals particularly as anti-asthmatic and anti-inflammatory compounds. In PCT International Application No. WO01/87281, certain PDE4 inhibitors are said to be useful in enhancing cognitive function.

Tully and Cavallieri disclose in US Patent Application Publication No. WO02/0076398 a method of therapy for cognitive deficits associated with a central nervous system disorder and methods of enhancing cognitive performance by combining cognitive training protocols with administration of CREB pathway-enhancing agents. Tully and Cavallieri does not disclose the use of this combination therapy for psychiatric disorders

Davis et al disclose in PCT International Application No. WO02/078629 methods for treating an individual with a psychiatric disorder with a pharmacologic agent that enhances learning or conditioning in combination with a session of psychotherapy. Davis et al does not disclose the use of PDE4 inhibitors in combination with psychotherapy.

Reines et al disclose in PCT International Application No. WO01/64223 the combination of a neurokinin-1 antagonist or an alpha-2 adrenoreceptor agonist with a PDE4 inhibitor for the treatment or prevention of depression and/or anxiety. Reines et al does not disclose the use of PDE4 inhibitors to augment the effect of psychotherapy.

In a second aspect, we have concluded that inhibition of phosphatase and PDE4, in conjunction with appropriate therapy is effective in the extinction of fear.

### SUMMARY OF THE INVENTION

The present invention provides a method for the treatment of psychiatric disorders using a phosphatase inhibitor in conjunction with psychotherapy. The inclusion of a phosphatase inhibitor (and optionally a PDE4 inhibitor) in the treatment modality enhances the effectiveness of psychotherapy resulting in fewer sessions required to achieve improvement, shorter intervals between sessions, or more pronounced improvement of symptoms, as compared to psychotherapy alone.

### DETAILED DESCRIPTION OF THE INVENTION

The present invention provides a method for the treatment of psychiatric disorder in a patient, said method comprises administering to said patient a therapeutically effective amount of a phosphatase inhibitor in combination with psychotherapy. The method comprises subjecting the individual to one or more sessions of a combination therapy protocol, where the combination therapy protocol comprises administering a therapeutically effective amount of a phosphatase inhibitor in combination with a session of psychotherapy.

Accordingly, in one embodiment, the invention is directed to method for the treatment of psychiatric disorder in a patient, said method comprises administering to said patient a therapeutically effective amount of a phosphatase inhibitor in combination with psychotherapy.

Within this embodiment, there is a genus wherein the psychiatric disorder is posttraumatic stress disorder.

Wherein this embodiment, there is another genus wherein the psychotherapy is selected from behavior psychotherapy, cognitive psychotherapy, and psychodynamically oriented psychotherapy.

Within this embodiment, there is another genus wherein the psychotherapy is exposure-based psychotherapy.

Within this embodiment there is another genus wherein the psychotherapy is cognitive psychotherapy.

Within this embodiment, there is another genus wherein the psychotherapy is structured as to enhance acquisition and consolidation, as opposed to enhance the ability forget undesired memories.

Within this embodiment, there is another genus wherein the phosphatase is calcineurin or PP1.

Within this embodiment, there is another genus wherein the method comprises administering to said patient a therapeutically effective amount of a calcineurin inhibitor in combination with psychotherapy.

Within this genus, there is a sub-genus wherein the calcineurin inhibitor is selected from Rapamycin and Tacrolimus.

As may be appreciated, in some classes of patients it may prove advantageous to have high levels of drug onboard at the time of the psychotherapy session. Thus, within this sub-genus, there is a class wherein the calcineurin inhibitor is administered prior to a session of said psychotherapy. Similarly, within the above embodiment there is a genus wherein the phosphatase inhibitor administered prior to a session of said psychotherapy.

Within this embodiment there is another genus further comprising the administration of a therapeutically effective amount of a PDE4 inhibitor.

Within this genus, there is a sub-genus wherein the PDE4 inhibitor is administered prior to a session of said psychotherapy.

Within this sub-genus, there is a class wherein the PDE4 inhibitor is selected from cilomilast, roflumilast and roflumilast N-oxide.

The inclusion of a phosphatase inhibitor (and optionally a PDE4 inhibitor) in the treatment modality enhances the effectiveness of psychotherapy resulting in fewer sessions required to achieve improvement, shorter intervals between sessions, or more pronounced improvement of symptoms, as compared to psychotherapy alone. In one aspect, it is envisioned that in the methods described herein, a single dose of the phosphatase inhibitor or the PDE-4 inhibitor or both will be administered in connection with a session of psychotherapy. For example, a single dose of a calcineurin inhibitor or a PDE-4 inhibitor or both may be taken 1 to 24 hours (e.g. 1, 2, 4, 6, 8, 12, 16 or 24 hours) before the session.

The term “psychiatric disorder,” as used herein, refers to a disorder that can be treated with the methods of the present invention. For purposes of the present invention, an individual said to have a psychiatric disorder will have one or more disorders that can be treated with the methods of the present invention. Thus an individual may have a single disorder, or may have a constellation of disorders that are to be treated by the methods described herein. The psychiatric disorders contemplated in the present invention include, but are not limited to, fear and anxiety disorders, addictive disorders including substance abuse disorders, and mood disorders. Within the fear and anxiety disorder category, the invention encompasses the treatment of panic disorder, specific phobia, posttraumatic stress disorder (PTSD), obsessive-compulsive disorder, and movement disorder such as Tourette's syndrome. The disorders contemplated herein are defined in, for example, the DSM-IV (Diagnostic and Statistical Manual, 4th edition, American Psychiatric Association).

In one aspect of the present invention, the psychiatric disorder to be treated is PTSD. Posttraumatic stress disorder (PTSD) is defined by DSM-IV as an anxiety disorder that an individual may develop following exposure to a traumatic event, and is characterized by (1) reexperiencing the traumatic event, such as recurrent nightmares, intrusive recollections of the event, flashbacks, physiological and psychological responses to internal or external cues relating to the event, etc; (2) persistent avoidance of thoughts, people or places associated with the event; (3) numbing of general responsiveness such as emotional detachment, restricted affect or loss of interest in activities; and (4) persistence of increased arousal such as exaggerated startle response, hypervigilence, irritability, difficulty sleeping, etc. In the US the lifetime prevalence of PTSD is at least 1%, and in high-risk populations, such as combat veterans or victims of criminal violence, prevalence is reported to be between 3 and 58%; PTSD is therefore of considerable public health concern.

The methods of the invention encompass the use of any type of psychotherapy that is suitable for the particular psychiatric disorder for which the individual is undergoing treatment, and may be conducted in one or more sessions. Suitable methods of psychotherapy include behavior psychotherapy such as exposure-based psychotherapy, cognitive psychotherapy including cognitive training and psychodynamically oriented psychotherapy (see, for example, Foa (2000) J. Clin. Psych. 61(suppl. 5):43-38). Exposure based psychotherapy include for example, systematic desensitization, flooding, implosive therapy, and extinction-based therapy. Such psychotherapy modalities are well known to one skilled in the art of psychiatry.

One method of psychotherapy specifically contemplated is the use of virtual reality (VR) exposure therapy to treat a psychiatric disorder using the combination therapy protocol of the invention. VR exposure therapy has been used to treat a variety of disorders including anxiety disorders such as the fear of heights (Rothbaum and Hodges (1999) Behav. Modif 23(4):507-25), as well as specific phobias, eating disorders, and PTSD (Anderson et al. (2001) Bull. Menninger Clin. 65(l):78-91). Because of the prevalence of PTSD in the general population and the successful use of VR therapy to treat PTSD in, for example, Vietnam veterans (Rothbaum et al. 30 (1999) J. Trauma Stress 12(2):263-71) or rape victims (Rothbaum et al. (2001) J. Trauma Stress 14(2):283-93), one embodiment of the present invention specifically contemplates the use of such VR exposure psychotherapy in combination with a PDE4 inhibitor as described elsewhere herein to treat PTSD.

The phosphatase inhibitors used to practice the present invention may be any that is known, or discovered to inhibit the phosphatase enzyme, and are not limited to any particular structural class of compounds. As used herein, the term “phosphatase inhibitors” includes any pharmaceutically acceptable salts thereof. The assay for identifying phosphatase inhibitors is described in the Examples section hereinbelow. The utility of phosphatase inhibitors in the present invention may be evaluated using the animal fear conditioning/extinction and clinical experimental protocols disclosed in PCT Application No. WO02/078629, which is hereby incorporated by reference, with the exception that a phosphatase inhibitor is used instead of the pharmacological agent used therein.

The phosphatase inhibitor may be peptidal or non-peptidal in nature; however, the use of a non-peptidal phosphatase inhibitor is preferred. In a preferred embodiment, the phosphatase inhibitor is a CNS-penetrant phosphatase inhibitor. In addition, for convenience the use of an orally active phosphatase inhibitor is preferred. To facilitate dosing, it is also preferred that the phosphatase inhibitor is a long acting phosphatase inhibitor. An especially preferred class of phosphatase inhibitors of use in the present invention are those compounds which are orally active and long acting. Representative phosphatase inhibitors of use in the present invention are fully described, for example, in U.S. Pat. Nos. 3,929,992; 4,894,366, 5,431,896; 5,208,228; 5,190,950; 5,532,248; 5,250,678; 5,565,560; 5,693,648; 5,247,076; 5,344,925; 5,252,732; 5,349,061; 5,550,233; 5,310,903; 5,091,389; 5,324,659; 5,318,895; 5,258,389; 5,310,901 which are hereby incorporated by reference, and in particular, Rapamycin and Tacrolimus.

The PDE4 inhibitors used to practice the present invention may be any that is known, or discovered to inhibit the PDE4 enzyme, and are not limited to any particular structural class of compounds. As used herein, the term “PDE4 inhibitors” includes any pharmaceutically acceptable salts thereof. The assay for identifying phosphatase inhibitors is described in the Examples section hereinbelow. The utility of PDE4 inhibitors in the present invention may be evaluated using the animal fear conditioning/extinction and clinical experimental protocols disclosed in PCT Application No. WO02/078629, which is hereby incorporated by reference, with the exception that a PDE4 inhibitor is used instead of the pharmacological agent used therein.

The PDE4 inhibitor may be peptidal or non-peptidal in nature; however, the use of a non-peptidal PDE4 inhibitor is preferred. In a preferred embodiment, the PDE4 inhibitor is a CNS-penetrant PDE4 inhibitor. In addition, for convenience the use of an orally active PDE4 inhibitor is preferred. To facilitate dosing, it is also preferred that the PDE4 inhibitor is a long acting PDE4 inhibitor. An especially preferred class of PDE4 inhibitors of use in the present invention are those compounds which are orally active and long acting. Representative PDE4 inhibitors of use in the present invention are fully described, for example, in U.S. Pat. Nos. 5,340,827, 5,550,137, 5,491,147, 5,608,070, 5,622,977, 5,633,257, 5,712,298, 5,739,144, 5,776,958, 5,780,477, 5,780,478, 5,786,354, 5,798,373, 5,580,888, 5,849,770, 5,859,034, 5,866,593, 5,891,896, 5,919,801, 6,005,118, 6,410,563, 6,399,639, 6,448,274 and International Patent Publications WO 94/22852, WO 95/35283, WO 96/00215. Suitable PDE4 inhibitors include pentoxifylline, isobutylmethylxanthine, cilomilast, roflumilast and its N-oxide, arofylline, lirimilast, GW84247, CP-671305, and terferol. Other suitable PDE4 inhibitors for use in the present invention are those presented in the Examples section and their pharmaceutically acceptable salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are benzenesulfonic, citric, hydrobromic, hydrochloric, maleic, phosphoric, sulfuric, and tartaric acids.

The inhibitor(s) of the invention may be administered to the patient prior to, during or after the psychotherapy session. It is preferably administered within about 24 hours prior to or following the session of psychotherapy, more preferably within about 24 hour prior to initiating psychotherapy, and even more preferably within about 12 hours prior to initiating psychotherapy. A full course of treatment of psychiatric disorder entails at least one session of this combination therapy protocol.

The The inhibitor(s) of the invention may be administered in a composition suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The The inhibitor(s) of the invention are administered in a therapeutically effective amount, which is that amount that provides improved therapeutic benefit relative to that achieved by psychotherapy alone. Dosage levels from about 0.001 mg/kg to about 140 mg/kg of body weight per day are useful for the purpose of the present invention or about 0.05 mg to about 7 g per patient per day. Alternatively, dosage levels from about 0.01 mg to 50 mg of the compound per kilogram of body weight per day, or alternatively about 0.5 mg to about 2.5 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 0.01 mg to about 1000 mg of the active ingredient, typically 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the The inhibitor(s) of the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the The inhibitor(s) of the invention may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of a compound of the Examples. The compounds or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices prepared via conventional processes As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an The inhibitor(s) of the invention may also be prepared in powder or liquid concentrate form.

Further, the compound of this invention can be utilized in combination with other therapeutic compounds. In particular, the combinations of the inhibitor(s) of the invention can be advantageously used in combination with i) Leukotriene receptor antagonists, ii) Leukotriene biosynthesis inhibitors, iii) COX-2 selective inhibitors, iv) statins, v) NSAIDs, vi) M2/M3 antagonists, vii) corticosteroids, viii) HI (histamine) receptor antagonists and ix) beta 2 adrenoceptor agonist.

Thus, for example, the inhibitor(s) of the invention may be administered with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered prior to, during or after a session of psychotherapy.

A subject undergoing treatment with the methods of the invention exhibits an improvement in one or more symptoms associated with the psychiatric disorder. For a description of the relevant symptoms, see, for example, the DSM-IV ((1994) Diagnostic and Statistical Mamlal of Mental Disorders (4th ea., American Psychiatric Association, Washington D.C.)), which is herein incorporated by reference. The efficacy of the methods of the invention can be assessed using any clinically recognized assessment method for measuring a reduction of one or more symptoms of the particular psychiatric disorder. Examples of such assessment methods are described in, for example, in Experiment 7 of PCT Application WO02/078629.

The present invention may be better understood with reference to the following examples. These examples are intended to be representative of specific embodiments of the invention, and are not intended as limiting the scope of the invention.

### EXAMPLES

### Spa (Scintilltion Proximity Assay) Based PDE Activity Assay Protocol

Compounds which inhibit the hydrolysis of cAMP to AMP by the type-IV cAMP-specific phosphodiesterases may be screened in a 96-well plate format as follows:

In a 96 well-plate at 30° C. is added the test compound (dissolved in 2 μL DMSO), 188 mL of substrate buffer containing [2,8-³H] adenosine 3′,5′-cyclic phosphate (cAMP, 100 nM to 50 μM), 10 mM MgCk₂, imM EDTA, 50 mM Tris, pH 7.5. The reaction is initiated by the addition of 10 mL of human recombinant PDE4 (the amount was controlled so that ˜10% product was formed in 10 min.). The reaction is stopped after 10 min. by the addition of 1 mg of PDE-SPA beads (Amersham Pharmacia Biotech, Inc., Piscataway, N.J.). The product AMP generated is quantified on a Wallac Microbeta® 96-well plate counter (EG&G Wallac Co., Gaithersburg, Md.). The signal in the absence of enzyme is defined as the background. 100% activity is defined as the signal detected in the presence of enzyme and DMSO with the background subtracted. Percentage of inhibition is calculated accordingly. IC₅₀ value is approximated with a non-linear regression fit using the standard 4-parameter/multiple binding sites equation from a ten point titration.

### COMPOUND EXAMPLES

The compound examples are comprised of two sub-sets—Example set A and Example set B,

The compounds of Examples 1A through 42A are characterized and prepared as disclosed in U.S. Pat. No. 6,410,563 B1, issued Jun. 25, 2002, which is hereby incorporated by reference.

1A. and 2A. 6-isopropyl-8-(3-{(Z/E)-2-[4-(methylsulfonyl)phenyl]-2-phenylethenyl}phenyl)quinoline;

3A. 6-isopropyl-8-{3-[(E/Z)-2-[4-(methylsulfonyl)phenyl]-2-(1,3-thiazol-2-yl)ethenyl]phenyl}quinoline;

4A. 6-isopropyl-8-(3-{(E)-2-(1-methyl-1H-imidazol-2-yl)-2-[4-(methylsulfonyl)phenyl]ethenyl}phenyl)quinoline;

5A. and 6A. 6-isopropyl-8-(3-{(Z/E)-2-(4-fluorophenyl)-2-[4-(methylsulfonyl)phenyl]ethenyl}phenyl)quinoline;

7A. 2-(2-{(E/Z)-2-[3-(6-isopropyl-8-quinolinyl)phenyl]-1-[4-(methylsulfonyl)phenyl]ethenyl}-1,3-thiazol-5-yl)-2-propanol;

8A. 2-[8-(3-{(E/Z)-2-[5-(1-hydroxy-1-methylethyl)-1,3-thiazol-2-yl]-2-[4-(methylsulfonyl)phenyl]ethenyl}phenyl)-6-quinolinyl]-2-methylpropanenitrile;

9A. 2-methyl-2-[8-(3-{(E)-2-(1-methyl-1H-imidazol-2-yl)-2-[4-(methylsulfonyl)phenyl]ethenyl}phenyl)-6-quinolinyl]propanenitrile;

10A. 6-[1-(methylsulfonyl)ethyl]-8-{3-[(E)-2-[4-(methylsulfonyl)phenyl]-2-(1,3-thiazol-2-yl)ethenyl]phenyl}quinoline;

11A. 6-[1-methyl-1-(methylsulfonyl)ethyl]-8-{3-[(E)-2-[4-(methylsulfonyl)phenyl]-2-(1,3-thiazol-2-yl)ethenyl]phenyl}quinoline;

12A. 8-(3-{(Z)-2-(1-methyl-1H-imidazol-2-yl)-2-[4-(methylsulfonyl)phenyl]ethenyl}phenyl)-6-[1-(methylsulfonyl)ethyl]quinoline;

13A. 8-(3-{(Z)-2-(1-methyl-1H-imidazol-2-yl)-2-[4-(methylsulfonyl)-phenyl]ethenyl}phenyl)-6-[1-methyl-1-(methylsulfonyl)ethyl]quinoline;

14A. and 15. 6-[1-methyl-1-(methylsulfonyl)ethyl]-8-(3-{(E/Z)-2-(3-methyl-1,2,4-oxadiazol-5-yl)-2-[4-(methylsulfonyl)phenyl]ethenyl}phenyl)quinoline;

16A. and 17A. (E/Z)-3-{3-[6-(1-cyano-1-methylethyl)-8-quinolinyl]phenyl)-N-isopropyl-2-[4-(methylsulfonyl)phenyl]-2-propenamide;

18A. 8-(3-{(E)-2-{3-[(4-methoxyphenoxy)methyl]-1,2,4-oxadiazol-5-yl}-2-[4-(methylsulfonyl)phenyl]ethenyl}phenyl)-6-[1-methyl-1-(methylsulfonyl)ethyl]quinoline;

19A. (5-{(E)-2-(3-{6-[1-methyl-1-(methylsulfonyl)ethyl]-8-quinolinyl}phenyl)-1-[4-(methylsulfonyl)phenyl)ethenyl}-1,2,4-oxadiazol-3-yl)methanol;

20A. (E)-N-isopropyl-3-(3-{6-[1-methyl-1-(methylsulfonyl)ethyl]-8-quinolinyl}phenyl)-2-[4-(methylsulfonyl)phenyl]-2-propenamide;

21A. (1)-3-{3-[6-(1-cyano-1-methylethyl)-8-quinolinyl]phenyl}-2-[4-(methylsulfonyl)phenyl]-2-propenoic acid;

22A. 2-methyl-2-[8-(3-{(E)-2-(3-methyl-1,2,4-oxadiazol-5-yl)-2-[4-(methylsulfonyl)phenyl]ethenyl}phenyl)-6-quinolinyl]propanenitrile;

23A. (E)-3-{3-[6-(1-cyano-1-methylethyl)-8-quinolinyl]phenyl}-2-[4-(methylsulfonyl)phenyl]-2-propenamide;

24A. (E)-N-(tert-butyl)-3-{3-[6-(1-cyano-1-methylethyl)-8-quinolinyl]phenyl}-2-[4-(methylsulfonyl)phenyl]-2-propenamide;

25A. (E)-3-[3-(6-isopropyl-8-quinolinyl)phenyl]-2-[4-(methylsulfonyl)phenyl]-2-propenoic acid;

26A. 6-isopropyl-8-(3-{(E)-2-(3-methyl-1,2,4-oxadiazol-5-yl)-2-[4-(methylsulfonyl)phenyl]ethenyl}phenyl)quinoline;

27A. (E)-3-(3-{6-[1-methyl-1-(methylsulfonyl)ethyl]-8-quinolinyl}phenyl)-2-[4-(methylsulfonyl)phenyl]-1-(1-pyrrolidinyl)-2-propen-1-one;

28A. (E)-N-cyclopropyl-3-(3-{6-[1-methyl-1-(methylsulfonyl)ethyl]-8-quinolinyl}phenyl)-2-[4-(methylsulfonyl)phenyl]-2-propenamide;

29A. (E)-N-(tert-butyl)-3-(3-{6-[1-methyl-1-(methylsulfonyl)ethyl]-8-quinolinyl}phenyl)-2-[4-(methylsulfonyl)phenyl]-2-propenamide;

30A. 8-{3-[2,2-bis(4-chlorophenyl)vinyl]phenyl}-6-isopropylquinoline;

31A. and 32A. 6-isopropyl-8-(3-{(E/Z)-2-(6-methyl-3-pyridinyl)-2-[4-(methylsulfonyl)phenyl]ethenyl}phenyl)quinoline;

33A. and 34A. 6-isopropyl-8-(3-{(E/Z)-2-(5-methyl-2-pyridinyl)-2-[4-(methylsulfonyl)phenyl]ethenyl}phenyl)quinoline;

35A. 8-(3-{2,2-bis[4-(methylsulfonyl)phenyl)vinyl}phenyl)-6-isopropylquinoline;

36A. and 37A. 2-methyl-2-[8-(3-{(E/Z)-2-(5-methyl-2-pyridinyl)-2-[4-(methylsulfonyl)phenyl]ethenyl}phenyl)-6-quinolinyl]propanenitrile;

38A. 2-[8-(3-{2,2-bis[4-(methylsulfonyl)phenyl]vinyl}phenyl)-6-quinolinyl]-2-methylpropanenitrile;

39A. 2-methyl-2-(8-{3-[(E)-2-[4-(methylsulfonyl)phenyl]-2-(2-pyridinyl)ethenyl]phenyl}-6-quinolinyl)propanenitrile;

40A. and 41A. 6-[1-methyl-1-(methylsulfonyl)ethyl]-8-(3-{(E/Z)-2-(5-methyl-2-pyridinyl)-2-[4-(methylsulfonyl)phenyl]ethenyl}phenyl)quinoline;

42A. 2-(6-{(E)-2-(3-{6-[1-methyl-1-(methylsulfonyl)ethyl]-8-quinolinyl}phenyl)-1-[4-(methylsulfonyl)phenyl]ethenyl}-3-pyridinyl)-2-propanol.

The compounds of Examples 1B through 32b are characterized and prepared as disclosed in U.S. Pat. No. 6,399,636 B2, issued Jun. 4, 2002, which is hereby incorporated by reference.
1B (±)-4{-2-[3,4-Bis(difluoromethoxy)phenyl]-2-{5-[2-(1-hydroxy-1-methyl)-ethyl]thiazolyl}ethyl}pyridine N-oxide;
2B chiral 4-{2-[3,4-Bis(difluoromethoxy)phenyl]-2-{5-[2-(1-hydroxy-1-methyl)ethyl]thiazolyl}ethyl}pyridine N-oxide;
3B (±/±)-4-{2-[3,4-Bis(difluoromethoxy)phenyl]-2-[5-(2-(1-hydroxy-2,2,2-trifluoro)ethyl)thiazolyl]ethyl}pyridine;
4B (±/±)-4-{2-[3,4-Bis(difluoromethoxy)phenyl]-2-[5-(2-(1-hydroxy-2,2,2-trifluoro)ethyl)thiazolyl]ethyl}pyridine N-oxide;
5B (±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine;
6B (±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine N-oxide;
7B (±/±)-4-{2-[3,4-Bis(difluoromethoxy)phenyl]-2-{5-[2-(1-hydroxy-1-trifluoromethyl)ethyl]thiazolyl}ethyl}pyridine N-oxide;
8B (±/±)-4-{2-[3,4-Bis(difluoromethoxy)phenyl]-2-[5-(2-phenylmethanol)-thiazolyl]ethyl}pyridine N-oxide;
9B (±/±)-4-{2-[3,4-Bis(difluoromethoxy)phenyl]-2-[5-(2-(1-hydroxy-1-phenyl)ethyl)thiazolyl]ethyl}pyridine N-oxide;
10B (±/±)-4-{2-[3,4-Bis(difluoromethoxy)phenyl]-2-[5-(2-(1-hydroxy-1-phenyl-2,2,2-trifluoro)ethyl)thiazolyl]ethyl}pyridine N-oxide;
11B (±/±)-4-{2-[3,4-Bis(difluoromethoxy)phenyl]-2-[5-(2-(1-hydroxy-1-phenyl)propyl)thiazolyl]ethyl}pyridine N-oxide;
12B (±/±)-4-{2-[3,4-Bis(difluoromethoxy)phenyl]-2-[5-(2-cyclohexylmethanol)-thiazolyl]ethyl}pyridine;
13B (±/±)-4-{2-[3,4-Bis(difluoromethoxy)phenyl]-2-[5-(2-(1-hydroxy-1-cyclohexyl-2,2,2-trifluoromethyl)ethyl)thiazolyl]ethyl}pyridine N-oxide;
14B (±/±)-4-{2-[3,4-Bis(difluoromethoxy)phenyl]-2-[5-(2-(1-hydroxy-1-(4-ethyl)phenyl)ethyl)thiazolyl]ethyl}pyridine N-oxide;
15B (±/±)-4-{2-[3,4-Bis(difluoromethoxy)phenyl]-2-[5-(2-(1-hydroxy-1-(4-ethyl)phenyl-2,2,2-trifluoro)ethyl)thiazolyl]ethyl}pyridine N-oxide;
16B (±/±)-4-{2-[3,4-Bis(difluoromethoxy)phenyl]-2-[5-(2-(1-hydroxy-1-(4-fluoro)phenyl)ethyl)thiazolyl]ethyl}pyridine N-oxide;
17B (±/±)-4-{2-[3,4-Bis(difluoromethoxy)phenyl]-2-[5-(2-(1-hydroxy-1-(4-fluoro)phenyl-2,2,2-trifluoro)ethyl)thiazolyl]ethyl}pyridine N-oxide;
18B (±/±)-4-{2-[3,4-Bis(difluoromethoxy)phenyl]-2-[5-(2-(1-hydroxy-1-(5-bromopyridin-2-yl)-2,2,2-trifluoro)ethyl)thiazolyl]ethyl}pyridine N-oxide;
19B (±/±)-4-{2-[3,4-Bis(difluoromethoxy)phenyl]-2-[5-(2-(1-hydroxy-1-(6-bromopyridin-3-yl)-2,2,2-trifluoro)ethyl)thiazolyl]ethyl}pyridine N-oxide;
20B (±)-4-{2-[3,4-Bis(difluoromethoxy)phenyl]-2-{5-[2-(1-hydroxy)cyclobutyl]-thiazolyl}ethyl}pyridine N-oxide;
21B (±)-4-{2-[3,4-Bis(difluoromethoxy)phenyl]-2-{5-[2-(1-hydroxy)cyclohexyl]-thiazolyl}ethyl}pyridine N-oxide;
22B (±)-4-{2-[(3-cyclobutyloxy-4-difluoromethoxy)phenyl]-2-{5-[2-(1-methyl)ethyl]thiazolyl}ethyl}pyridine N-oxide;
23B chiral 4-{2-[(3-cyclobutyloxy-4-difluoromethoxy)phenyl]-2-{5-[2-(1-hydroxy-1-methyl)ethyl]thiazolyl}ethyl}pyridine N-oxide;
24B (±)-4-{2-[(3-cyclobutyloxy-4-difluoromethoxy)phenyl]-2-{5-[2-(1-trifluoromethyl-2,2,2-trifluoro)ethyl]thiazolyl}ethyl}pyridine;
25B (±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
26B Chiral 3-{2-[(3-cyclobutyloxy-4-difluoromethoxy)phenyl]-2-{5-[2-(1-hydroxy-1-methyl)ethyl]thiazolyl}ethyl}pyridine;
27B Chiral 3-{2-[(3-cyclobutyloxy-4-difluoromethoxy)phenyl]-2-{5-[2-(1-hydroxy-1-methyl)ethyl]thiazolyl}ethyl}pyridine N-oxide;
28B Chiral 3-{2-[(3-cyclobutyloxy-4-difluoromethoxy)phenyl]-2-{5-[2-(1-hydroxy-1-methyl)ethyl]thiazolyl}ethyl}pyridine N-oxide;
29B Chiral 3-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
30B (±)-4-{2-[(3-cyclopropyloxy-4-difluoromethoxy)phenyl]-2-{5-[2-(1-hydroxy-1-methyl)ethyl]thiazolyl}ethyl}pyridine N-oxide;
31B (±)-3-{2-[(3-cyclopropyloxy-4-difluoromethoxy)phenyl]-2-{5-[2-(1-hydroxy-1-trifluoro methyl-2,2,2-trifluoro)ethyl]thiazolyl}ethyl}pyridine N-oxide; and
32B chiral 3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide.

Additional PDE4 inhibitors within the scope of the invention are prepared as follows:

### NAPHTHYRIDINONE 1

### N-Cyclopropyl-1-(3-bromophenyl)-1,4-dihydro[1,8]naphthyridin-4-one-3-carboxamide

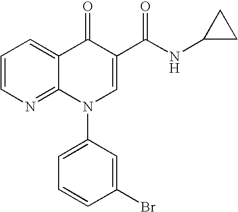

### Step 1: Ethyl 3-(3-bromoanilino)-2-(2-chloronicotinoyl)acrylate

A mixture of ethyl 2-chloronicotinoyl acetate (prepared following a procedure described in J. Het. Chem., 30, 855, 1993) (1 eq), triethyl orthoformate (1.5 eq) and acetic anhydride (5 eq) was heated at 130° C. for 2.5 hours. The volatile components were distilled off and the residue was co-evaporated twice with xylene. The oily residue was dissolved in methylene chloride and 3-bromoaniline (1.2 eq) was added slowly. The resulting solution was stirred at room temperature for 18 hours, and the solvent evaporated away. The resulting crude compound was used as such in the next step.

### Step 2: Ethyl 1-(3-bromophenyl)-1,4-dihydro[1,8]naphthyridin-4-one-3-carboxylate

The crude compound from Step 1 was dissolved in tetrahydrofuran (0.3 M), the solution was cooled to 0° C., and sodium hydride (as a 60% dispersion in oil, 1.3 eq) was added in portions. After stirring at 0° C. for 1 hour, the mixture was allowed to warm up to room temperature. After 2 hours, water was added to the suspension and and the insoluble solid was filtered and washed copiously with water. When dry, the solid was stirred in ether at room temperature for 24 hours and filtered to afford the title compound as a cream-colored solid.

¹H NMR (Acetone-d₆) δ 1.32 (t, 3H), 4.29 (q, 2H), 7.54-7.63 (m, 2H), 7.69 (dd, 1H), 7.78 (dd, 1H), 7.93 (s, 1H), 8.66-8.71 (m, 3H).

Alternatively, the following procedure for step 1 to 2 can be used:

A mixture of 2-chloronicotinoyl chloride (1 eq), triethylamine (4 eq) and ethyl 3,3-dimethylaminoacrylate (1.5 eq) in acetonitrile (0.5M) was heated to reflux for 3 h, cooled to 40-50° C. and 3-bromoaniline (1 eq) was added. The reaction was heated to reflux overnight, cooled to rt, diluted with water (2 volume). The product was isolated by filtration and washed with water, ether or acetonitrile-water (1:1).

### Step 3: 1-(3-Bromophenyl)-1,4-dihydro[1,8]naphthyridin-4one-3-carboxylic acid

A suspension of ethyl 1-(3-bromophenyl)-1,4dihydro[1,8]naphthyridin-4-one-3-carboxylate from Step 2 (1 eq) in a mixture of tetrahydrofuran-methanol (0.15M) and 1N aqueous sodium hydroxide (2 eq) was heated at ca 50° C. with stirring for 20 minutes. After cooling, the mixture was diluted with water and acidified with 1N aqueous HCl. After stirring for 45 minutes, the precipitate was filtered, washed well with water and dried to afford the title acid as a cream-colored solid.

¹H NMR (Acetone-d₆) δ 7.65 (t, 1H), 7.76 (m, 2H), 7.84 (d, 1H), 7.99 (s, 1H), 8.87 (m, 2H), 9.01 (s, 1H).

### Step 4: N-Cyclopropyl-1-(3-bromophenyl)-1,4-dihydro[1,8]naphthyridin-4-one-3-carboxamide

To a suspension of 1-(3-bromophenyl)-1,4-dihydro[1,8]naphthyridin-4-one-3-carboxylic acid from Step 3 (1 eq) and triethylamine (3 eq) in tetrahydrofuran (0.08M) at 0° C. was added isobutyl chloroformate (1.8 eq). After stirring at 0° C. for 2 hours, cyclopropylamine (5 eq) was added and the mixture was allowed to warm up to room temperature and stirred overnight. The mixture was then partitioned between ethyl acetate and water, the organic phase was dried and evaporated to a solid which was stirred in ether at room temperature for 3 hours and filtered to afford the N-Cyclopropyl-1-(3-bromophenyl)-1,4-dihydro[1,8]naphthyridin-4-one-3-carboxamide.

¹H NMR (Acetone-d₆) δ 0.59 (m, 2H), 0.80 (m, 2H), 2.96 (m, 1H), 7.59-7.68 (m, 2H), 7.72 (dd, 1H), 7.82 (dd, 1H), 7.97 (s, 1H), 8.72-8.81 (m, 2H), 8.89 (s, 1H), 9.70 (br, NH).

### NAPHTHYRIDINONE 2

### N-Cyclopropyl-4-oxo-1-[3-(4,4,5,5-tetramethyl-1,3,2-dioxaborolan-2-yl)phenyl]-1,4-dihydro-1,8-naphthyridine-3-carboxamide

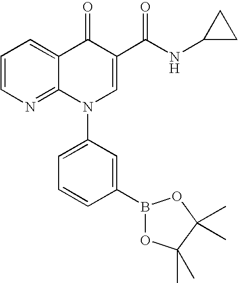

A mixture of NAPHTHYRIDINONE 1 (1.0 eq), pinacol diborane (1.5 eq), KOAc (4 eq) and PdCl₂(dppf) (0.05 eq) in DMP (0.2M) was stirred at 70-80° C. for 3 h. The mixture was cooled to rt, diluted with EtOAc and a NH₄Cl solution. The organic extracts were washed with H₂O, brine, dried over MgSO₄, filtered and concentrated. Crystallization from ether and flash chromatography (CH₂Cl₂:EtOAc, 50:50) of the mother liquor afforded the title compound as a white solid.

¹H NMR (500 MHz, acetone-d₆): δ 9.78 (s, 1H), 8.90 (s, 1H), 8.79 (dd, 1H), 8.72 (dd, 1H), 7.94 (d, 1H), 7.91 (s, 1H), 7.80 (d, 1H), 7.69 (t, 1H), 7.62 (dd, 1H), 2.9 (m, 1H), 1.38 (s, 12H), 0.80 (m, 2H), 0.60 (m, 2H).

### EXAMPLE 1C

### 2-(trans)-{3′-[3-[(Cyclopropylamino)carbonyl]-4-oxo-1,8-naphthyridin-1(4H)-yl]-1,1′-biphenyl-4-yl}cyclopropanecarboxylic acid

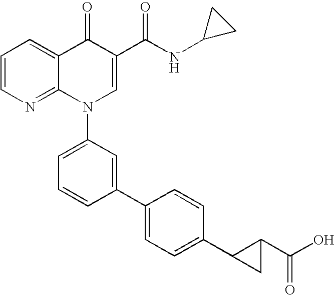

### Step 1: Ethyl 2-(trans)-(4-bromophenyl)cyclopropanecarboxylate

To a mixture of ethyl 4-bromocinnamate and Pd(OAc)₂ (0.05 eq) in methylene chloride (1M) at 0° C. was added dropwise a solution of CH₂N₂ in ether until the reaction was completed by NMR analysis. The mixture was filtered through a plug of silica gel and concentrated to afford the title compound as an oil.

### Step 2: Ethyl 2-(trans)-[4-(4,4,5,5-tetramethyl-1,3,2-dioxaborolan-2-yl)phenyl]cyclopropanecarboxylate

A mixture of bromide from step 1 (1.0 eq), pinacol diborane ester (1.4 eq), KOAc (3.5 eq) and PdCl₂(dppf)₂ (0.03 eq) in DMF (0.14M) was stirred at 60° C. for 24 h. The resulting mixture was cooled to rt, diluted with EtOAc:hexane (1:1). The organic phase was washed with water (3×), brine, dried over MgSO₄, filtered and concentrated. Flash chromatography (hexane:EtOAc; 90:10) afforded the title compound.

### Step 3: 2-(trans)-[4-(4,4,5,5-Tetramethyl-1,3,2-dioxaborolan-2-yl)phenyl]cyclopropanecarboxylic acid

A mixture of ester from step 2 and NaOH (20%, 30 mL) was heated to 100° C. for 1.5 h, cooled to rt, acidified with HCl 10% and extracted with EtOAc. The organic extract was dried over Na₂SO₄ and the solvent evaporated to afford the title compound.

### Step 4: 2-(trans)-{3′-[3-[(Cyclopropylamino)carbonyl]-4-oxo-1,8-naphthyridin-1(4H)-yl]-1,1′-biphenyl-4-yl}cyclopropanecarboxylic acid

A mixture of NAPHTHYRIDINONE 1(1.0 eq), acid from step 3 (1.5 eq), Na₂CO₃ (3.5 eq; 2M in H₂O), Pd(OAc)₂ (0.05 eq.) and PPh₃ (0.15 eq.) or PdCl₂dppf (0.05 eq) in n-propanol-DMF (1:1, 0.1M) was stirred at 70° C. for 2 h. The mixture was cooled to rt, quenched with AcOH and diluted with EtOAc. The combined organic extracts were washed with brine, dried over Na₂SO₄, filtered and concentrated. Flash chromatography (CH₂Cl₂:EtOAc, 60:40, 2% AcOH) afforded the title compound as a white solid.

¹H NMR (500 MHz, CDCl₃): δ 9.9 (d, 1H), 9.08 (s, 1H), 8.77 (dd, 1H), 8.69 (dd, 1H), 7.71 (d, 1H), 7.60 (m, 2H), 7.52 (d, 2H), 7.45 (m, 1H), 7.38 (d, 1H), 7.13 (d, 2H), 2.97 (m, 1H), 2.54 (m, 1H), 1.87 (m, 1H), 1.60 (m, 1H), 1.35 (m, 1H), 0.85 (m, 2H), 0.65 (m, 2H). MS(H⁻):464.2

The optically active isomers of EXAMPLE 1C can be isolated separately by chromatography using chiral column; for example Chiral Pak AD eluting with hexane:EtOH or hexane:iPrOH containing 0.2% TFA.

Alternatively, separation can be achieved on intermediate (trans)-2-(4-bromophenyl)cyclopropanecarboxylic acid

### (trans)-2-(4-Bromophenyl)cyclopropanecarboxylic acid

To a solution of ester from step 1 in THF-MeOH (4:1, 0.5M) was added LiOH (3 eq, 2M) and the mixture was stirred at 50° C. for 1 h. The organic solvent evaporated, aqueous was acidified with HCl 1N and the acid extracted with EtOAc (3×). The organic was washed with brine, dried and solvent evaporated to afford 2-(4-bromophenyl)cyclopropanecarboxylic acid. Optically active precursors are obtained by separation on chiral column (Chiral Pak AD) eluting with hexane:EtOH or hexane:iPrOH containing 0.2% TFA.

Another alternative is using a chiral auxiliary as follows,

### Step 1: (trans)-3-(4-Bromo-phenyl)-1-imidazol-1-yl-propenone

To a solution of (trans)-3-(4-bromo-phenyl)acrylic acid (1.0 eq) in toluene (0.2M) was added CDI (1.5 eq). The mixture was stirred for 3 h at rt. The resulting precipitate was isolated by filtration to afford the title compound as a white solid.

### Step 2: (trans)-3-[3-(4-Bromo-phenyl)-acryloyl]-4-methyl-5-phenyl-oxazolidin-2-one

A mixture of 3-(4-bromo-phenyl)-1-imidazol-1-yl-propenone (1.05 eq) from Step 1, (4R, 5S)-(+)-4-methyl-5-phenyl-2-oxazolidinone (1.0 eq) or (−)-isomer and Et₃N (1.2 eq) in CH₃CN (0.2M) was refluxed overnight. The resulting mixture was cooled to rt, filtered on a pad of silica gel and concentrated. Crystallization in hexane:Et₂O afforded the title compound as a white solid.

### Step 3: (trans)-3-[2-(4-Bromo-phenyl)cyclopropanecarbonyl]-4-methyl-5-phenyl-oxazolidin-2-one

To a solution of (trans)-3-[3-(4-bromo-phenyl)-acryloyl]-4-methyl-5-phenyl-oxazolidin-2-one from Step 2 and Pd(OAc)₂ (0.05 eq.) in THF (0.2M) was added portionwise CH₂N₂ until the reaction was completed. NMR of aliquots monitored the reaction. The resulting mixture was concentrated and flash chromatography (Hex:EtOAc; 3:2) to afford the two separate diastereoisomers. Each diastereoisomer were submitted separately to next procedures to afford the (+) and (−) enantiomers of EXAMPLE 1C.

### Step 4: 2-(trans)-(4-Bromophenyl)cyclopropanecarboxylic acid

To a solution of amide from step 3 in THF-EtOH—H₂O (4:1:1, 0.1M) was added LiOH (2.4 eq, 2M) and the mixture was stirred at rt for 2 h. The mixture was neutralized to pH 7 with HCl 1N, the organic solvent evaporated and the resulting residue dissolved in ether. The organic was washed with NaOH 1N (2×). The combined aqueous layers were acidified and extracted with ether (3×). The organic extract was washed brine, dried and solvent evaporated to afford (+) or (−)-(trans)-2-(4-bromophenyl)cyclopropanecarboxylic acid as a white solid.

### Step 5: 2-(trans)-{3′-[3-[(Cyclopropylamino)carbonyl]-4-oxo-1,8-naphthyridin-1(4H)-yl]-1,1′-biphenyl-4yl}cyclopropanecarboxylic acid

A mixture of NAPHTHYRIDINONE 2 (1.0 eq), 2-(trans)-(4-bromophenyl)cyclopropanecarboxylic acid (1.2 eq.) from step 4, Na₂CO₃ (3.5 eq; 2M in H₂O), Pd(OAc)₂ (0.05 eq.) and PPh₃ (0.15 eq) in n-propanol (0.1M) was stirred at 70° C. for 4 h. The mixture was cooled to rt, poured in water and extracted with EtOAc (2×). The combined organic extracts were washed with brine, dried over Na₂SO₄, filtered and concentrated. Flash chromatography (CH₂Cl₂:EtOAc, 50:50, 2% AcOH) afforded the title compound as a white solid.

Another alternative is an enantioselective cyclopropanation using for example a bis-oxazoline chiral ligand/copper complex and diazoacetate (Evans et al. J. Am. Chem. Soc. 1991, 113, 726) to prepare optically active ethyl 2-(trans)-(4-bromophenyl)cyclopropanecarboxylate from 4-bromostyrene. Selective hydrolysis of the mixture of cis and trans isomer with LiOH (1 eq based on the trans ester) gave 2-(trans)-(4-bromophenyl)cyclopropanecarboxylic acid and ethyl 2-(cis)-(4-bromophenyl)cyclopropanecarboxylate which can be used in EXAMPLE 4.

### EXAMPLE 2C

### 2-{3′-[3-[(Cyclopropylamino)carbonyl]-4-oxo-1,8-naphthyridin-1(4H)-yl]-1,1′-biphenyl-4-yl}-2-methylpropanoic acid

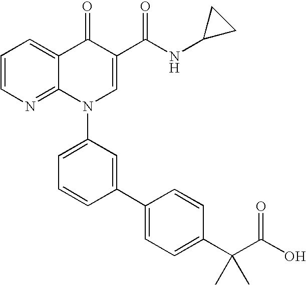

### Step 1: Methyl 2-(4-bromophenyl)-2-methylpropanoate

To a solution of LiHMDS (1M, THF, 2.1 eq) in THF (0.06M) was added methyl 4-bromophenylacetate (1 eq). After 15 min, MeI (4 eq) was added and the reaction mixture slowly warmed to rt and stirred for 18 h. The mixture was quenched with HCl 10%, diluted with EtOAc, washed with HCl 10%, brine, dried and solvent evaporated. Flash chromatography (Hexane:EtOAc:, 95:5) afforded the title compound.

### Step 2: 2-(4-Bromophenyl)-2-methylpropanoic acid

To a solution of ester from step 1 in THF-MeOH (4:1, 0.5M) was added LiOH (3 eq, 2M) and the mixture was stirred at 50° C. for 1 h. The organic solvent evaporated, aqueous was acidified with HCl 1 N and the acid extracted with EtOAc (3×). The organic was washed with brine, dried and solvent evaporated to afford the acid.

### Step 3: 2-{3′-[3-[(Cyclopropylamino)carbonyl]-4-oxo-1,8-naphthyridin-1(4H)-yl]-1,1′-biphenyl-4-yl}-2-methylpropanoic acid

A mixture of NAPHTHYRIDINONE 2 (1.0 eq), acid from step 2 (1.5 eq), Na₂CO₃ (3.5 eq.; 2M in H₂O) and Pd(Ph₃)₄ or PdCl2(dppf) or Pd(OAc)₂(Ph3P)₃ (0.05 eq) in n-propanol (0.1M) was stirred at 70-90° C. for 3 h. The mixture was cooled to rt, quenched with HCl and diluted with EtOAc. The combined organic extracts were washed with brine, dried over MgSO₄, filtered and concentrated. Flash chromatography (CH₂Cl₂:EtOAc, 40:60, 2% AcOH) afforded the title compound as a white solid.

¹H NMR (500 MD, DMSO-d₆): δ 9.75 (d, 1H), 8.81 (s, 1H), 8.77 (m, 1H), 8.71 (d, 1H), 7.92 (s, 1H), 7.86 (d, 1H), 7.70 (d, 2H), 7.70-7.55 (m, 3H), 7.45 (d, 2H), 2.88 (m, 1H), 1.50 (s, 6H), 0.77 (m, 2H), 0.55 (m, 2H).

### EXAMPLE 3C

### 3-{3′-[3-[(Cyclopropylamino)carbonyl]-4-oxo-1,8-naphthyridin-1(4H)-yl]-1,1′-biphenyl-4-yl}-3-methylbutanoic acid

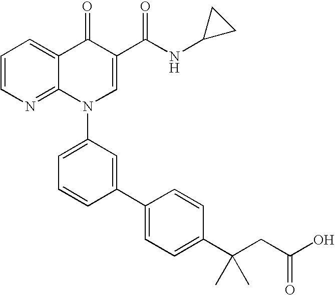

Prepared according to the procedure described in EXAMPLE 2C, step 2 and 3 but using 3-(4-iodo-phenyl)-3-methyl-butyric acid methyl ester (prepared according to the procedure described in J. Am. Chem. Soc. 1948, 70, 370) as starting material. Flash chromatography (CH₂Cl₂:EtOAc, 40:60, 2% AcOH) afforded the title compound as a white solid.

¹H NMR (500 MHz, DMSO-d₆): δ 9.75 (d, 1H); 8.82 (s, 1H), 8.78 (dd, 1H), 8.72 (dd, 1H), 7.94 (s, 1H), 7.86 (d, 1H), 7.68-7.60 (m, 2H), 7.66 (d, 2H), 7.57 (d, 1H), 7.49 (d, 2H), 2.88 (m, 1H), 260 (s, 2H), 1.40 (s, 6H), 0.76 (m, 2H), 0.55 (m, 2H).

### EXAMPLE 4C

### 2-(cis)-{3′-[3-[(Cyclopropylamino)carbonyl]-4-oxo-1,8-naphthyridin-1(4H)-yl]-1,1′-biphenyl-4-yl}cyclopropanecarboxylic acid

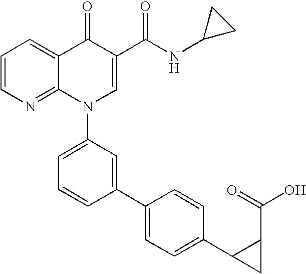

### Step 1: Methyl 2-(cis)-3-(4-bromophenyl)prop-2-enoate

To a solution of bis(trifluoroethyl)(methoxycarbonylmethyl)phosphonate and 18-Crown-6 (5 eq) in THF (0.05M) at −78° C. was added KHMDS (1 eq, 0.5M, toluene) followed by 4-bromobenzaldehyde (1 eq). The reaction mixture was stirred at −78° C. 1 h, quenched with a saturated ammonium chloride solution and diluted with ether. The combined organic extracts were washed with brine, dried over Na₂SO₄, filtered and concentrated. Flash chromatography (Hexane:EtOAc; 9:1 to 7:3) afforded the title compound.

### Step 2: Methyl 2-(cis)-(4bromophenyl)cyclopropanecarboxylate

To a mixture of ester from step 1 and Pd(OAc)₂ (0.05 eq) in methylene chloride (1M) at 0° C. was added dropwise a solution of CH₂N₂ in ether until the reaction was completed by NMR analysis. Flash chromatography (Hexane:EtOAc; 100:0 to 90:10) afforded the title compound.

### Step 3: Methyl 2-(3′-[3-[(cyclopropylamino)carbonyl]-4-oxo-1,8-naphthyridin-1(4H)-yl]-1,1′-biphenyl-4-yl}cyclopropanecarboxylate

Prepared according to the procedure described in EXAMPLE 2C, step 3 but using ester from Example 4, step 2 as starting material. The product was purified by flash chromatography (hexane:EtOAc, 60:40), then vigorous stirring in hexane/ether and isolation by filtration to afford the title compound as a white solid.

### Step 4: 2-(cis)-{3′-[3-[(Cyclopropylamino)carbonyl]-4-oxo-1,8-naphthyridin-1(4H)-yl]-1,1′-biphenyl-4-yl}cyclopropanecarboxylic acid

To a solution of ester in THF-EtOH (1:1, 0.05M) was added LiOH (5 eq, 2M) and the mixture was stirred at 60° C. for 4 h. The mixture was cooled to rt, extracted with ether. The aqueous was acidified with HCl 1N and the acid extracted with EtOAc (3×). The organic was washed with brine, dried and solvent evaporated. The residue was stirred vigorously in CH₂Cl₂/hexanelacetone and isolated by filtration to afford the title compound as an off-white solid.

¹H NMR (500 MHz, DMSO-d₆): δ 11.88 (br s, 1H), 9.75 (d, 1H), 8.80 (s, 1H), 8.79 (dd, 1H), 8.74 (dd, 1H), 7.95 (s, 1H), 7.87 (d, 1H), 7.63-7.70 (m, 4H), 7.59 (d, 1H), 7.35 (d, 2H), 2.89-2.93 (m, 1H), 2.62 (dd, 1H), 2.04-2.09 (m, 1H), 1.31-1.35 (m, 1H), 0.77-0.87 (m, 2H), 0.56-0.59 (m, 2H).

The optically active diastereoisomers of EXAMPLE 4C can be isolated separately by chromatography using chiral column; for example Chiral Pak AD eluting with hexane:EtOH or hexane:iPrOH containing 0.2% TFA.

Alternatively, the optically active intermediate can be obtained as follow:

### (cis)-2-(4-Bromophenyl)cyclopropanecarboxylic acid

To a solution of ester from step 2 in THF-MeOH (4:1, 0.5M) was added LiOH (3 eq, 2M) and the mixture was stirred at 50° C. for 1 h. The organic solvent was evaporated, the aqueous layer was acidified with HCl 1N and the acid extracted with EtOAc (3×). The combined organic extracts were washed with brine, dried and evaporated to afford (cis)-2-(4-bromophenyl)-cyclopropanecarboxylic acid. Optically active intermediates were obtained by separation on chiral column (Chiral Pak AD) using hexane:EtOH (90:10 with 0.2% TFA)

Another alternative is using chiral auxiliary as follows,

### Step 1: (cis)-1-{[2-(4-Bromophenyl)cyclopropyl]carbonyl}-1H-imidazole

To a solution of (cis)-2-(4-bromophenyl)cyclopropanecarboxylic acid (1.0 eq.) in toluene (0.2M) was added CDI (1.5 eq.). The mixture was stirred for 18 h at 50° C. The solvent evaporated, the resulting residue stirred vigorously in hexane/CH₂Cl₂, filtered, the filtrate evaporated to afford the title compound as a white solid.

### Step 2: (cis)-3-{[2-(4-Bromophenyl)cyclopropyl]carbonyl}-4-methyl-5-phenyl-1,3-oxazolidin-2-one

A mixture of (cis)-1-{[2-(4-bromophenyl)cyclopropyl]carbonyl}-1H-imidazole (1 eq) from Step 1, (4R, 5S)-(+)-4-methyl-5-phenyl-2-oxazolidinone (1.2 eq) or (−)-isomer and DBU (added at 0° C., 1.2 eq) in CH₃CN (0.2M) was stirred at 0° C. for 4 h. The solvent evaporated and residue purified by flash chromatography (Hexane:EtOAc; 100:0.to 80:20) to afford each diastereoisomer.

### Step 3: (cis)-2-(4-Bromophenyl)cyclopropanecarboxylic acid

To a solution of amide (either (+) or (−) isomer) from step 2 above) in THF-H₂O (4:1, 0.5M) at 0° C. was added LOH (1.6 eq, 2M) and H₂O₂ (35%, 4 eq). The mixture was stirred at 0° C. for 4 h. The organic solvent was evaporated, the mixture extracted with CH₂Cl₂, the aqueous was acidified with HCl 1N and the acid extracted with EtOAc (3×). The combined organic extracts were washed with brine, dried and solvent evaporated to afford either (+) or (−) optically active (cis)-2-(4-bromophenyl)cyclopropanecarboxylic acid.

Another alternative is an enantioselective cyclopropanation using for example a bis-oxazoline chiral ligand/copper complex and diazoacetate (Evans et al. J. Am. Chem. Soc. 1991, 113, 726) to prepare optically active ethyl 2-(cis)-(4-bromophenyl)cyclopropanecarboxylate from 4-bromostyrene. Selective hydrolysis of the mixture of cis and trans isomer with LiOH (1 eq based on trans isomer) gave 2-(trans)-(4-bromophenyl)cyclopropanecarboxylic acid (used in EXAMPLE 1) and the desired ethyl 2-(cis)-(4-bromophenyl)cyclopropanecarboxylate.

### EXAMPLE 5C

### 1-{3′-[3-[(Cyclopropylamino)carbonyl]-4-oxo-1,8-naphthyridin-1(4H)-yl]-1,1′-biphenyl-4-yl}cyclopropanecarboxylic acid

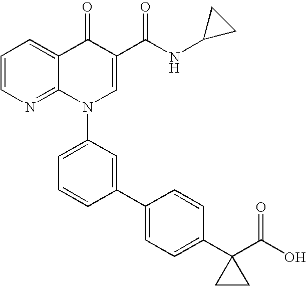

### Step 1: 1-(4-Bromophenyl)cyclopropanecarbonitrile

A mixture of 4-bromophenylacetonitrile (1 eq), 1,2-bromochloroethane (1.5 eq), benzyltriethylammonium chloride (0.14 eq) in NaOH 50% (4M) was heated to 50° C. for 18 h. The mixture was cooled to rt, quenched with HCl 5% and diluted with ether. The combined organic extracts were washed with brine, dried over MgSO₄, filtered and concentrated. Flash chromatography (hexane:EtOAc, 95:5) afforded the title compound.

### Step 2: 1-(4-Bromophenyl)cyclopropanecarboxylic acid

A mixture of nitrile (1 eq) from step 1, NaOH 25% (12 eq) in EtOH (0.5M) was heated to 100° C. for 5 h. The mixture was cooled to rt, quenched with AcOH and diluted with ether. The combined organic extracts were washed with brine, dried over MgSO₄, filtered and concentrated. Flash chromatography (hexane:EtOAc, 90:10 to 50:50) afforded the title compound.

### Step 3: 1-{3′-[3-[(Cyclopropylamino)carbonyl]-4-oxo-1,8-naphthyridin-1(4H)-yl]-1,1′-biphenyl-4-yl}cyclopropanecarboxylic acid

Prepared according to the procedure described in EXAMPLE 2C, step 3 but using acid from step 2 as starting material. Flash chromatography (CH₂Cl₂:MeOH, 98:2) afforded the title compound as a white solid.

¹H NMR (500 MHz, acetone-d₆): δ 9.92 (d, 1H), 9.77 (d, 1H), 8.96 (s, 1H), 8.79 (dd, 1H), 8.74 (dd, 1H), 8.00 (s, 1H), 7.76-7.72 (m, 3H), 7.65 (dd, 1H), 7.61 (dd, 1H), 7.52 (d, 2H), 2.98-2.94 (m, 1H), 1.60-1.58 (m, 2H), 1.25-1.22 (m, 2H), 0.82-0.78 (m, 2), 0.62-0.59 (m, 2H).

### EXAMPLE 6C

### (trans)-2-{3′-[3-[(Cyclopropylamino)carbonyl]-4-oxo-1,8-naphthyridin-1(4H)-yl]-3-fluoro-1,1′-biphenyl-4-yl}cyclopropanecarboxylic acid

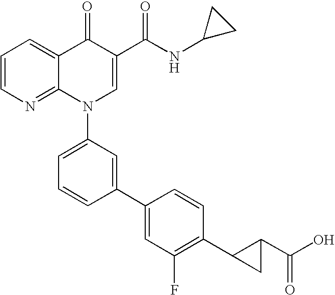

### Step 1: Methyl(trans)-2-(4-bromo-2-fluorophenyl)cyclopropanecarboxylate

To a mixture of 4-bromo-2-fluorocinnamic acid and Pd(OAc)₂ (0.05 eq) in methylene chloride (1M) at 0° C. was added dropwise a solution of CH₂N₂ in ether until the reaction was completed by NMR analysis. The residue was purified by flash chromatography (hexane:EtOAc, 100:0 to 70:30).

### Step 2: Methyl(trans)-2-{3′-[3-[(cyclopropylamino)carbonyl]-4-oxo-1,8-naphthyridin-1(4H)-yl]-3-fluoro-1,1′-biphenyl-4-yl}cyclopropanecarboxylate

A mixture of NAPHTHYRIDINONE 2 (1.0 eq), ester from step 1 (1.3 eq), Na₂CO₃ (3.5 eq; 2M in H₂O), Pd(OAc)₂ (0.05 eq) and PPh₃ (0.15 eq) or PdCl₂dppf (0.05 eq) in n-propanol (0.1M was stirred at 60-80° C. for 1-3 h. The mixture was cooled to rt, poured in water and extracted with EtOAc (2×). The combined organic extracts were washed with brine, dried over Na₂SO₄, filtered and concentrated. Flash chromatography (CH₂Cl₂:EtOAc; 90:10) afforded the title compound.

### Step 3: 2-(trans)-{3′-[3-[(Cyclopropylamino)carbonyl]-4-oxo-1,8-naphthyridin-1(4H)-yl]-fluoro-1,1′-biphenyl-4-yl}cyclopropanecarboxylic acid

To a solution of ester from above step 2 in THF-MeOH (2:1, 0.2M) was added LiOH (5 eq, 2M) and the mixture was stirred at rt for 6 h. The organic solvent evaporated, aqueous was acidified with AcOH and the acid extracted with EtOAc (3×). The organic was washed with brine, dried and solvent evaporated. The residue was triturated in CH₂Cl₂/ether and then isolated by filtration to afford the title compound as a solid.

¹H NMR (500 MHz, CDCl₃): δ 9.85 (d, 1H), 9.08 (s, 1H), 8.80 (dd, 1H), 8.70 (dd, 1H), 7.71 (d, 1H), 7.63 (t, 1H), 7.60 (s, 1H), 7.46 (dd, 1H), 7.40 (d, 1H), 7.28 (m, 2H), 7.01 (t, 1H), 2.97 (m, 1H), 2.66 (m, 1H), 1.93 (m, 1H), 1.62 (m, 1H), 1.40 (m, 1H), 0.84 (m, 2H), 0.66 (m, 2H).

Optically active (+) or (−)-(trans)-2-{3′-[3-[(cyclopropylamino)carbonyl]-4-oxo-1,8-naphthyridin-1(4H)-yl]-3-fluoro-1,1′-biphenyl-4-yl}cyclopropanecarboxylic acid was obtained using optically active (+) or (−)-(trans)-methyl 2-(4-bromo-2-fluorophenyl)cyclopropanecarboxylate prepared according to procedure described below:

### Step A: 4-bromo-2-fluoro-1-vinylbenzene

To a suspension of methyltriphenylphosphonium bromide (1.1 eq) in tetrahydrofuran (0.13M) at −78° C., was added n-butyllithium (2.5M in hexanes, 1.1 eq) dropwise over 20 min. The reaction mixture was stirred at −78° C. for 15 min, warm to 0° C., stirred for 15 min and cooled back to −78° C. A solution of 4-bromo-2-fluoro-benzaldehyde (1 eq) in 100 mL THF was added dropwise over 30 min. Final mixture was allowed to warm slowly to rt and stirred for 1 h. The resulting mixture was quenched with a saturated NH₄Cl solution and diluted with 2 volume of hexane. The organic extract was washed with brine, dried over MgSO₄, filtered and concentrated. The residue was diluted with Hexane/EtOAc (9:1) and filtered on a pad of Silica gel. The fractions were combined and concentrated to afford the desired material

### Step B: (+)-ethyl 2-(4-bromo-2-fluorophenyl)cyclopropanecarboxylate

To a suspension of copper(I) trifluoromethanesulfonate benzene complex (0.01 eq) in chloroform (100 mL) at rt, was added (R,R)-2,2′-isopropylidene-bis(4-tert-butyl-2-oxazoline) (0.01 eq). The mixture was stirred at for 1 h then cannulated through glass wool in a solution of styrene from step 5 (1.2 eq) in chloroform (0.1M) at 4° C. To this solution was added a solution of ethyl diazoacetate (0.8 eq) in of chloroform (1M) dropwise over 3 h. The final mixture was stirred overnight at 4° C. The mixture was concentrated. Flash chromatography (Hexane, EtOAc; 95:5) afforded the desired compound. (84:16, trans:cis isomers, methodology described in Evans et al. J. Am. Chem. Soc. 1991, 113, 726)

### Step C: (+)-2-(4-bromo-2-fluorophenyl)cyclopropanecarboxylic acid

To a solution of (+)-ethyl 2-(4-bromo-2-fluorophenyl)cyclopropanecarboxylate (mix esters, 1 eq) in tetrahydrofuran-methanol (2:1) was added lithium hydroxide (0.84 eq). The reaction was stirred at rt for 2 days. The resulting mixture was concentrated, diluted with water, extracted with ether(2×) to obtain the cis ester. The aqueous phase was acidified using HCl 10%, extracted with ether (2×) to obtain the (+)-trans acid. The organic extract containing the trans acid were combined and washed with brine, dried over MgSO₄, filtered and concentrated.

### Step D: (+)-methyl 2-(4-bromo-2-fluorophenyl)cyclopropanecarboxylate

To a solution of (+)-2-(4-bromo-2-fluorophenyl)cyclopropanecarboxylic acid from step C (1 eq) in methylenechloride was added an ethereal solution of diazomethane until reaction is completed by TLC. The resulting mixture was concentrated. The crude optically active ester was used as such in step 2 above.

### EXAMPLE 7C

### 2-(trans)-{3-Chloro-3′-[3-[(cyclopropylamino)carbonyl]-4-oxo-1,8-naphthyridin-1(4H)-yl]-1,1′-biphenyl-4-yl)cyclopropanecarboxylic acid

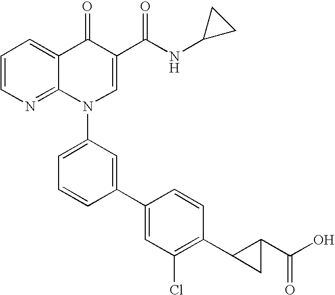

### Step 1: Ethyl 2-(trans)-3-(4-bromo-2-chlorophenyl)prop-2-enoate

To a solution of 4-bromo-2-chlorobenzaldehyde (1 eq) and triethylphosphonoacetate (1.1 eq) in THF (0.3M) at rt was added dropwise potassium t-butoxide (1.1 eq, 1M, THF). The mixture was stirred at rt 3 h, quenched with HCl 10%, diluted with ether, washed with a NaHCO₃ solution, brine, dried over MgSO₄, filtered and concentrated. Flash chromatography (Hexane:EtOAc; 90:10 to 70:30) afforded the title compound.

### Step 2: 2-(trans)-{3-Chloro-3′-[3-[(cyclopropylamino)carbonyl]-4-oxo-1,8-naphthyridin-1(4H)-yl]-1,1′-biphenyl-4-yl}cyclopropanecarboxylic acid

Prepared according to the procedure described in EXAMPLE 1C, but using ester from step 1 as starting material.

¹H NMR (500 MHz, CDCl₃): δ 9.88 (s, 1H), 9.10 (s, 1H), 8.80 (d, 1H), 8.70 (d, 1H), 7.72 (d, 1H), 7.65 (m, 3H), 7.45 (m, 3H), 7.05 (d, 1H), 2.95 (m, 1H), 2.70 (m, 1H), 1.80 (m, 1H), 1.65 (m, 1H), 1.35 (m, 1H), 0.85 (m, 2H), 0.65 (m, 2H).

### EXAMPLE 8C

### 2-(cis)-{3′-[3-[(Cyclopropylamino)carbonyl]-4-oxo-1,8-naphthyridin-1(4H)-yl]-3-fluoro-1,1′-biphenyl-4-yl}cyclopropanecarboxylic acid

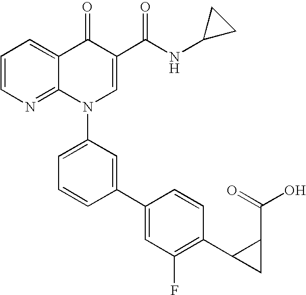

Prepared according to the procedure described in EXAMPLE 4C, but using 4-bromo-2-fluorobenzaldehyde as starting material.

¹H NMR (500 MHz, DMSO-d₆): δ 11.91 (br s, 1H), 9.75 (d, 1H), 8.83 (s, 1H), 8.79 (dd, 1H), 8.74 (dd, 1H), 8.02 (s, 1H), 7.93 (d, 1H), 7.70 (t, 1H), 7.62-7.66 (m, 2H), 7.57 (d, 1H), 7.54 (d, 1H), 7.36 (t, 1H), 2.88-2.94 (m, 1H), 2.55 (q, 1H), 2.08-2.13 (m, 1H), 1.53 (dd, 1H), 1.36-1.40 (m, 1H), 0.77-0.81 (m, 2H), 0.56-0.59 (m, 2H).

### EXAMPLE 9C

### 2-(trans)-{3′-[4-Oxo-3-{[(2,2,2-trifluoroethyl)amino]carbonyl}-1,8-naphthyridin-1(4H)-yl]-1,1′-biphenyl-4-yl}cyclopropanecarboxylic acid

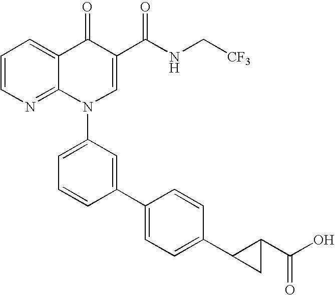

### Step 1: 1-(3-bromophenyl)-4-oxo-N-(2,2,2-trifluoroethyl)-1,4-dihydro-1,8-naphthyridine-3-carboxamide

A solution of acid from NAPHTHYRIDINONE 1, step 3 (1 eq) in DMF (0.1M) was added HATU (4 eq), diisopropylethylamine (8 eq) followed by 2,2,2-trifluoroethanamine (4 eq). The mixture was stirred at rt for 12 h then heated at 80° C. for 2 h, cooled to rt and diluted with water. The residue then isolated by filtration washed with ether, triturated in acetone to afford the title compound.

### Step 2: 2-(trans)-{3′-[4-Oxo-3-{[(2,2,2-trifluoroethyl)amino]carbonyl}-1,8-naphthyridin-1(4H)-yl]-1,1′-biphenylyl}cyclopropanecarboxylic acid

Prepared according to the procedure described in EXAMPLE 1C, but using amide from step 1 as starting material.

¹H NMR (500 MHz, Cl₃): δ 11.3 (s, OH), 10.34 (s, NH), 9.11 (s, 1H), 8.88 (dd, 1H), 8.77 (dd, 1H), 7.78 (d, 1H), 7.68 (t, 1H), 7.65 (s, 1H), 7.56 (d, 2H), 7.52 (dd, 1H), 7.44 (dd, 1H), 7.22 (d, 2H), 4.21 (br qt, 2H), 2.62 (m, 1H), 1.97 (m, 1H), 1.70 (m, 1H), 1.45 (m, 1H), MS+ESI Q1 (M+1) 508.1.

### EXAMPLE 10C

### (+)-(trans)-2-{3-fluoro-3′-[4-oxo-3-{[(2,2,2-trifluoroethyl)amino]carbonyl}-1,8-naphthyridin-1(4H)-yl)biphenyl-4-yl}cyclopropanecarboxylic acid

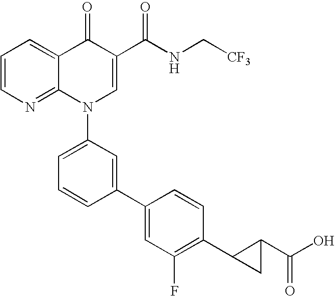

### Step 1: Ethyl 1-(3-bromophenyl)-1,4-dihydro[1,8]naphthyridin-4-one-3-carboxylate

A mixture of ethyl 2-chloronicotinoyl acetate (purchased or prepared following a procedure described in J. Het. Chem., 30, 855, 1993) (1 eq), triethylamine (4 eq) and ethyl 3,3-dimethylaminoacrylate (1.5 eq) in acetonitrile (0.5M) was heated to reflux for 3 h, cooled to 40-50° C. and 3-bromoaniline (1 eq) was added. The reaction was heated to reflux overnight, cooled to rt, diluted with water (2 volume). The product was isolated by filtration and washed with water, ether or acetonitrile-water (1:1).

¹H NMR (Acetone-d₆) δ 1.32 (t, 3H), 4.29 (q, 2H), 7.54-7.63 (m, 2H), 7.69 (dd, 1H), 7.78 (dd, 1H), 7.93 (s, 1H), 8.66-8.71 (m, 3H).

### Step 2: 1-(3-Bromophenyl)-1,4-dihydro[1,8]naphthyridin-4-one-3-carboxylic acid

A suspension of ethyl 1-(3-bromophenyl)-1,4-dihydro[1,8]naphthyridin-4-one-3-carboxylate from Step 1 (1 eq) in a mixture of tetrahydrofuran-methanol (0.15M) and 1N aqueous sodium hydroxide (2 eq) was heated at ca 50° C. with stifling for 20 minutes. After cooling, the mixture was diluted with water and acidified with 1N aqueous HCl. After stirring for 45 minutes, the precipitate was filtered, washed well with water and dried to afford the title acid as a cream-colored solid.

¹H NMR (Acetone-d₆) δ 7.65 (t, 1H), 7.76 (m, 2H), 7.84 (d, 1H), 7.99 (s, 1H), 8.87 (m, 2H), 9.01 (s, 1H).

### Step 3: 1-(3-bromophenyl)-N-(2,2,2-trifluoroethyl)-1,4-dihydro-1,8-naphthyridin-4-one-3-carboxamide

To a suspension of 1-(3-bromophenyl)-1,4-dihydro[1,8]naphthyridin-4-one-3-carboxylic acid from Step 2 (1 eq) and triethylamine (3 eq) in tetrahydrofuran (0.08M) at 0° C. was added isobutyl chloroformate (1.8 eq). After stirring at 0° C. for 2 hours, 2,2,2-trifluoroethylamine (5 eq) was added and the mixture was allowed to warm up to room temperature and stirred overnight. The mixture was then partitioned between ethyl acetate and water, the organic phase was dried and evaporated to a solid which was stirred in ether at room temperature for 3 hours and filtered to afford the N-(2,2,2-trifluoroethyl)-1-(3-bromophenyl)-1,4-dihydro[1,8]naphthyridin-4-one-3-carboxamide as a white solid.

Alternatively:

A suspension of 1-(3-bromophenyl)-1,4-dihydro[1,8]naphthyridin-4-one-3-carboxylic acid from Step 2 (1 eq) in DMF (0.1M) was added HATU (4 eq), diisopropylethylamine (8 eq) followed by 2,2,2-trifluoroethylamine (4 eq). The mixture was stirred at rt for 12 h then heated at 80° C. for 2 h, cooled to rt and diluted with water. The residue then isolated by filtration washed with ether, triturated in acetone to afford the title compound.

### Step 4: N-(2,2,2-trifluoroethyl)-1-[3-(4,4,5,5-tetramethyl-1,3,2-dioxaborolan-2-yl)phenyl]-1,4-dihydro-1,8-naphthyridin-4-one-3-carboxamide

A mixture of 1-(3-bromophenyl)-N-(2,2,2-trifluoroethyl)-1,4-dihydro-1,8-naphthyridin-4-one-3-carboxamide (1.0 eq), pinacol diborane (1.5 eq), KOAc (4 eq) and PdCl₂(dppf) (0.05 eq) in DMF (0.2M) was stirred at 70-80° C. for 3 h. The mixture was cooled to rt, diluted with EtOAc and a NH₄Cl solution. The organic extracts were washed with H₂O, brine, dried over MgSO₄, filtered and concentrated. Crystallization from EtOAc-Ether-Hexane (1:1:2) and flash chromatography (CH₂Cl₂:EtOAc, 50:50) of the mother liquor afforded the title compound as a white solid.

### Step 5: 4-bromo-2-fluoro-1-vinylbenzene

To a suspension of methyltriphenylphosphonium bromide (1.1 eq) in tetrahydrofuran (0.13M) at −78° C., was added n-butyllithium (2.5M in hexanes, 1.1 eq) dropwise over 20 min. The reaction mixture was stirred at −78° C. for 15 min, warm to 0° C., stirred for 15 min and cooled back to −78° C. A solution of 4-bromo-2-fluoro-benzaldehyde (1 eq) in 100 mL THF was added dropwise over 30 min. Final mixture was allowed to warm slowly to rt and stirred for 1 h. The resulting mixture was quenched with a saturated NH₄Cl solution and diluted with 2 volume of hexane. The organic extract was washed with brine, dried over MgSO₄, filtered and concentrated. The residue was diluted with Hexane/EtOAc (9:1) and filtered on a pad of Silica gel. The fractions were combined and concentrated to afford the desired material

### Step 6: (+)-ethyl 2-(4-bromo-2-fluorophenyl)cyclopropanecarboxylate

To a suspension of copper(I) trifluoromethanesulfonate benzene complex (0.01 eq) in chloroform (100 mL) at rt, was added (R,R)-2,2′-isopropylidene-bis(4-tert-butyl-2-oxazoline) (0.01 eq). The mixture was stirred at for 1 h then cannulated through glass wool in a solution of styrene from step 5 (1.2 eq) in chloroform (0.1M) at 4° C. To this solution was added a solution of ethyl diazoacetate (0.8 eq) in of chloroform (1M) dropwise over 3 h. The final mixture was stirred overnight at 4° C. The mixture was concentrated. Flash chromatography (Hexane, EtOAc; 95:5) afforded the desired compound. (84:16, trans:cis isomers, methodology described in Evans et al. J. Am. Chem. Soc. 1991, 113, 726)

### Step 7: (+)-2-(4-bromo-2-fluorophenyl)cyclopropanecarboxylic acid

To a solution of (+)-ethyl 2-(4-bromo-2-fluorophenyl)cyclopropanecarboxylate (mix esters, 1 eq) in tetrahydrofuran-methanol (2:1) was added lithium hydroxide (0.84 eq). The reaction was stirred at rt for 2 days. The resulting mixture was concentrated, diluted with water, extracted with ether(2×) to obtain the cis ester. The aqueous phase was acidified using HCl 10%, extracted with ether (2×) to obtain the (+)-trans acid. The organic extract containing the trans acid were combined and washed with brine, dried over MgSO₄, filtered and concentrated.

### Step 8: (+)-methyl 2-(4-bromo-2-fluorophenyl)cyclopropanecarboxylate

To a solution of (+)-2-(4-bromo-2-fluorophenyl)cyclopropanecarboxylic acid from step 7 (1 eq) in methylenechloride was added an ethereal solution of diazomethane until reaction is completed by TLC. The resulting mixture was concentrated. The crude ester was used as such in the next step.

### Step 9: (+)-methyl 2-{3-fluoro-3′-[4-oxo-3-{[(2,2,2-trifluoroethyl)amino]carbonyl}-1,8-naphthyridin-1(4H)-yl]biphenyl-4-yl}cyclopropanecarboxylate

To a solution of (+)-methyl 2-(4-bromo-2-fluorophenyl)cyclopropanecarboxylate from step 8 (1.2 eq) and boronate ester from step 4 (1 eq) in DMF-iPrOH (1:1, 0.1M), was added tris(dibenzylideneacetone)dipalladium(0) (0.055 eq), 2-(dimethylamino)-2′-(dicyclohexylphosphino)biphenyl (0.13 eq) and a sodium carbonate solution (2M, 4 eq) The reaction mixture was heated at 78° C. for 2 h. The reaction mixture was filtered on Celite and silica gel (1:1) and washed with EtOAc. The filtrate was concentrated in vacuo and remaining solvents were distilled in vacuo. The resulting yellow solid is stirred vigourously in ether. The residue then isolated by filtration and washed with ether. Mother liquors were further purified by flash chromatography (toluene/EtOAc, 100:0 to 70:30).

### Step 10: (+)-2-{3-fluoro-3′-[4-oxo-3-{[(2,2,2-trifluoroethyl)amino]carbonyl}-1,8-naphthyridin-1(4H)-yl]biphenyl-4-yl}cyclopropanecarboxylic acid

To a solution of ester from step 9 in THF-MeOH—H₂O (7:3:1, 0.05M) was added LiOH (1.1 eq, 1M) and the mixture was stirred at 50° C. for 3 h, then at room temperature for 24 h. The organic solvent evaporated, aqueous was acidified with HCl 1N and the acid extracted with EtOAc (3×). The organic was washed with brine, dried and solvent evaporated to afford 2-(3-bromophenyl)cyclopropanecarboxylic acid. The resulting solid is stirred vigorously in hexane-ether-acetone for 1 h then collected by filtration.

The enantiomer, (−)-2-{3-fluoro-3′-[4-oxo-3-{[(2,2,2-trifluoroethyl)amino]carbonyl}-1,8-naphthyridin-1(4H)-yl]biphenyl-4-yl}cyclopropanecarboxylic acid can be obtained using (S,S)-2,2′-isopropylidene-bis(4-tert-butyl-2-oxazoline) in step 6.

¹H NMR (500 MHz, CDCl₃): δ 10.31 (s, NH), 9.10 (s, 1H), 8.9 (dd, 1H), 8.78 (m, 1H), 7.78 (dd, 1H), 7.71 (t, 1H), 7.64 (br s, 1H), 7.53 (m, 1H), 7.47 (dd, 1H), 7.35 (m, 2H), 7.09 (t, 1H), 4.16 (m, 2H), 3.77 (m, 1H), 2.00 (m, 1H), 1.71 (m, 1H), 1.52 (m, 1H). MS+ESI Q1 (M+1) 525.9

### EXAMPLE 11C

### 1-({3′-[3-[(cyclopropylamino)carbonyl]-4-oxo-1,8-naphthyridin-1(4H)-yl]biphenyl-4-yl}methyl)cyclobutanecarboxylic acid

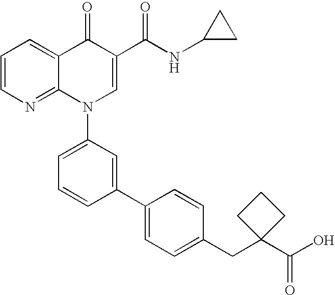

### Step 1: ethyl 1-(4-bromobenzyl)cyclobutanecarboxylate

To a solution of IDA (1.2 eq) in THF at −78° C. was added ethyl cyclobutanecarboxylate (1 eq) and reaction mixture was warmed up to 40° C. for 10 min. The reaction was cooled back to −78° C. for 1 h, then 4-bromobenzyl bromide (1.1 eq) was added as a solid to the mixture. The reaction mixture was warmed to 0° C. in an ice bath. The reaction was quenched with a saturated solution of NH₄Cl and extract 3× with ethyl acetate, dried with MgSO₄ and evaporated to dryness. Flash chromatography with 95:5 hexanes:ethyl acetate afforded ethyl 1-(4-bromobenzyl)cyclobutanecarboxylate.

### Step 2: ethyl 1-({3′-[3-[(cyclopropylamino)carbonyl]4-oxo-1,8-naphthyridin-1(4H)-yl]biphenyl-4-yl}methyl)cyclobutanecarboxylate

A mixture of NAPHTHYRIDINONE 2 (1.0 eq), ester from step 1 (1.5 eq), Na₂CO₃ (3.5 eq; 2M in H₂O), Pd(OAc)₂ (0.05 eq.) and PPh₃ (0.15 eq.) or PdCl₂dppf (0.05 eq) in n-propanol-DMF (1:1, 0.1M) was stirred at 70° C. for 2 h. The mixture was cooled to rt, quenched with AcOH and diluted with EtOAc. The combined organic extracts were washed with brine, dried over Na₂SO₄, filtered and concentrated. Flash chromatography (CH₂Cl₂/MeOH, 99:1) afforded the title compound.

### Step 3: 1-({3′-[3-[(cyclopropylamino)carbonyl]-4-oxo-1,8-naphthyridin-1(4H)-yl]biphenyl-4-yl}methyl)cyclobutanecarboxylic acid

To a solution of ester from above step 2 in THF-MeOH—H₂O (2:1:0.5, 0.1M) was added LiOH (5 eq, 2M) and the mixture was stirred at 50° C. for 4 h. The organic solvent evaporated, aqueous was acidified with AcOH and the acid extracted with EtOAc (3×). The organic was washed with brine, dried and solvent evaporated. The residue was purified by flash chromatography (CH₂Cl₂/MeOH, 10% NH₄OH, 85:15) to afford the title compound as a white solid.

¹H NMR (500 MHz, acetone-d₆): δ 9.87 (s, NH), 9.1 (s, 1H), 8.84 (dd, 1H), 8.72 (dd, 1H), 7.72 (d, 1H), 7.61 (m, 2H), 7.49 (m, 3H), 7.39 (d, 1H), 7.27 (m, 1H), 3.18 (s, 2H), 3.05 (m, 1H), 2.5 (m, 2H), 2.12 (m, 2H), 1.95 (m, 2H), 0.90 (m, 2H), 0.70 (m, 2H). MS 492.0 (−)

The following compounds were prepared according to the procedures described previously. Indicated is their respective (M+1)⁺ value obtained from a low resolution mass spectrometer under electron-spray or chemical ionization conditions. * indicate a (M−1)-value.

| | | LRMS |
|---|---|---|
| EX. | Chemical name | (M + 1)⁺ |
| 12C | (trans)-2-{3′-[3-[(cyclopropylamino)carbonyl]-4-oxo 1,8- | 480.2 |
| | naphthyridin-1(4H)-yl]biphenyl-4-yl}-2- | |
| | methylcyclopropanecarboxylic acid | |
| 13C | 3-methyl-3-{3′-[4-oxo-3-{[(2,2,2-trifluoroethyl)amino]carbonyl}- | 524.3 |
| | 1,8-naphthyridin-1(4H)-yl]biphenyl-4-yl}butanoic acid | |
| 14C | (+)-(trans)-2-{3-chloro-3′-[3-[(cyclopropylamino)carbonyl]-4-oxo | 500.2 |
| | 1,8-naphthyridin-1(4H)-yl]biphenyl-4-yl}cyclopropanecarboxylic | |
| | acid | |
| 15C | (−)-(trans)-2-{3′-[4-oxo-3-{[(2,2,2-trifluoroethyl)amino]carbonyl}- | 506.2* |
| | 1,8-naphthyridin-1(4H)-yl]biphenyl-4-yl}cyclopropanecarboxylic | |
| | acid | |
| 16C | 3-{3′-[3-[(cyclopropylamino)carbonyl]-4-oxo-1,8-naphthyridin- | 482.2 |
| | 1(4H)-yl]biphenyl-4-yl}-2,2-dimethylpropanoic acid | |
| 17C | 4-{3′-[3-[(cyclopropylamino)carbonyl]-4-oxo-1,8-naphthyridin- | 496.2 |
| | 1(4H)-yl]biphenyl-4-yl}-3,3-dimethylbutanoic acid | |
| 18C | 1-({3′-[4-oxo-3-{[(2,2,2-trifluoroethyl)amino]carbonyl}-1,8- | 534.2* |
| | naphthyridin-1(4H)-yl]biphenyl-4-yl}methyl)cyclobutanecarboxylic | |
| | acid | |
| 19C | 2,2-dimethyl-4-{3′-[4-oxo-3-{[(2,2,2- | 538.2 |
| | trifluoroethyl)amino]carbonyl}-1,8-naphthyridin-1(4H)- | |
| | yl]biphenyl-4-yl}butanoic acid | |
| 20C | 2,2-dimethyl-3-{3′-[4-oxo-3-{[(2,2,2- | 524.4 |
| | trifluoroethyl)amino]carbonyl}-1,8-naphthyridin-1(4H)- | |
| | yl]biphenyl-4-yl}propanoic acid | |

| | |
|---|---|
| 12C | 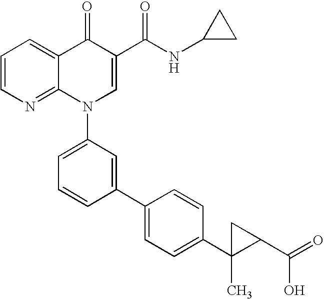 |
| 13C | 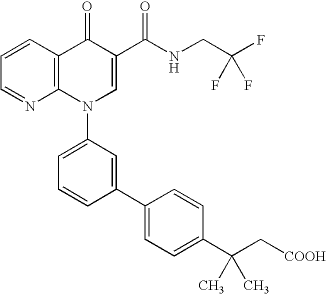 |
| 14C | 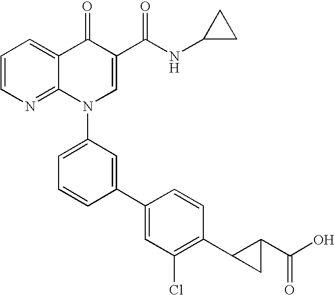 |
| 15C | 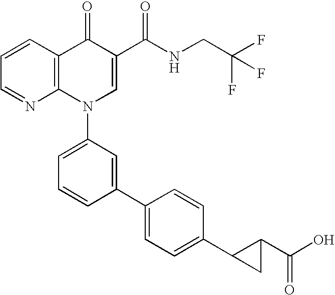 |
| 16C | 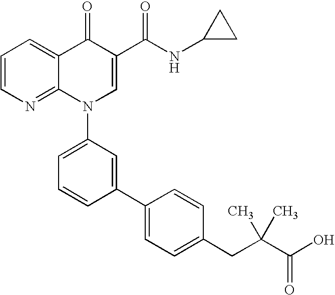 |
| 17C | 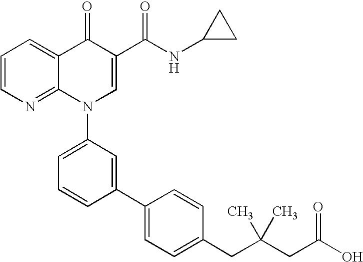 |
| 18C | 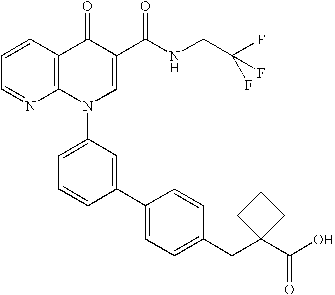 |
| 19C | 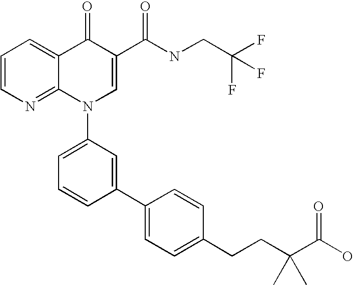 |
| 20C | 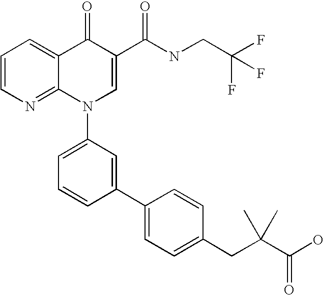 |

Other variations or modifications, which will be obvious to those skilled in the art, are within the scope and teachings of this invention. This invention is not to be limited except as set forth in the following claims.

What is claimed is:

1. A method for the treatment of psychiatric disorder in a patient, said method comprises administering to said patient a therapeutically effective amount of a phosphatase inhibitor in combination with psychotherapy.

2. The method of claim 1 wherein said psychiatric disorder is posttraumatic stress disorder.

3. The method of claim 1 wherein said psychotherapy is selected from behavior psychotherapy, cognitive psychotherapy, and psychodynamically oriented psychotherapy.

4. The method of claim 2 wherein said psychotherapy is selected from behavior psychotherapy, cognitive psychotherapy, and psychodynamically oriented psychotherapy.

5. The method of claim 1 wherein said psychotherapy is cognitive or exposure-based psychotherapy.

6. The method of claim 2 wherein said psychotherapy is cognitive or exposure-based psychotherapy.

7. The method of claim 1 wherein said psychotherapy is cognitive psychotherapy.

8. The method of claim 2 wherein said psychotherapy is cognitive psychotherapy.

9. The method of claim 1 wherein said psychotherapy is structured as to enhance acquisition and consolidation of new memories, as opposed to enhance the ability forget undesired memories.

10. The method of claim 1 wherein the phosphatase inhibitor is administered prior to a session of said psychotherapy.

11. The method of claim 1 wherein the phosphatase inhibitor is administered up to 24 hours prior to a session of said psychotherapy.

12. The method of claim 1 wherein said phosphatase is calcineurin or PP1.

13. The method for the treatment of psychiatric disorder in a patient, according to claim 1, said method comprises administering to said patient a therapeutically effective amount of a calcineurin inhibitor in combination with psychotherapy.

14. The method of claim 13 wherein said calcineurin inhibitor is selected from Rapamycin and Tacrolimus.

15. The method according to claim 13, wherein said calcineurin inhibitor is administered prior to a session of said psychotherapy.

16. The method according to claim 15, wherein said calcineurin inhibitor is administered up to 24 hours prior to a session of said psychotherapy.

17. The method of claim 13, further comprising the administration of a therapeutically effective amount of a PDE4 inhibitor.

18. The method according to claim 17, wherein said PDE4 inhibitor is administered prior to a session of said psychotherapy.

19. The method according to claim 18, wherein said PDE4 inhibitor is administered up to 24 hours prior to a session of said psychotherapy.

20. The method of claim 17 wherein said PDE4 inhibitor is selected from cilomilast, roflumilast and roflumilast N-oxide.

21. The method of claim 17 wherein said PDE4 inhibitor is selected from the group consisting of:
6-isopropyl-8-(3-{(Z/E)-2-[4-(methylsulfonyl)phenyl]-2-phenylethenyl}phenyl)quinoline;
6-isopropyl-8-{3-[(E/Z)-2-[4-(methylsulfonyl)phenyl]-2-(1,3-thiazol-2-yl)ethenyl]-phenyl}quinoline;
6-isopropyl-8-(3-{(E)-2-(1-methyl-1H-imidazol-2-yl)-2-[4-(methylsulfonyl)phenyl]ethenyl}-phenyl)quinoline;
6-isopropyl-8-(3-{(Z/E)-2-(4-fluorophenyl)-2-[4-(methylsulfonyl)phenyl]ethenyl}-phenyl)quinoline;
2-(2-{(E/Z)-2-[3-(6-isopropyl-8-quinolinyl)phenyl]-1-[4-(methylsulfonyl)phenyl]ethenyl}-1,3-thiazol-5-yl)-2-propanol;
2-[8-(3-{(E/Z)-2-[5-(1-hydroxy-1-methylethyl)-1,3-thiazol-2-yl]-2-[4-(methylsulfonyl)-phenyl]ethenyl}phenyl)-6-quinolinyl]-2-methylpropanenitrile;
2-methyl-2-[8-(3-{(E)-2-(1-methyl-1H-imidazol-2-yl)-2-[4-(methylsulfonyl)phenyl]ethenyl}-phenyl)-6-quinolinyl]propanenitrile;
6-[1-(methylsulfonyl)ethyl]-8-{3-[(E)-2-[4-(methylsulfonyl)phenyl]-2-(1,3-thiazol-2-yl)-ethenyl]phenyl}quinoline;
6-[1-methyl-1-(methylsulfonyl)ethyl]-8-{3-[(E)-2-[4-(methylsulfonyl)phenyl]-2-(1,3-thiazol-2-yl)ethenyl]phenyl}quinoline;
8-(3-{(Z)-2-(1-methyl-1H-imidazol-2-yl)-2-[4-(methylsulfonyl)phenyl]ethenyl}phenyl)-6-[1-(methylsulfonyl)ethyl]quinoline;
8-(3-{(Z)-2-(1-methyl-1H-imidazol-2-yl)-2-[4-(methylsulfonyl)phenyl]ethenyl}phenyl)-6-[1-methyl-1-(methylsulfonyl)ethyl]quinoline;
6-[1-methyl-1-(methylsulfonyl)ethyl]-8-(3-{(E/Z)-2-(3-methyl-1,2,4-oxadiazol-5-yl)-2-[4-(methylsulfonyl)phenyl]ethenyl}phenyl)quinoline;
(E/Z)-3-{3-[6-(1-cyano-1-methylethyl)-8-quinolinyl]phenyl}-N-isopropyl-2-[4-(methylsulfonyl)phenyl]-2-propenamide;
8-(3-{(E)-2-{3-[(4-methoxyphenoxy)methyl]-1,2,4-oxadiazol-5-yl}-2-[4-(methyl-sulfonyl)phenyl]ethenyl}phenyl)-6-[1-methyl-1-(methylsulfonyl)ethyl]quinoline;
(5-{(E)-2-(3-{6-[1-methyl-1-(methylsulfonyl)ethyl]-8-quinolinyl}phenyl)-1-[4-(methylsulfonyl)phenyl]ethenyl}-1,2,4-oxadiazol-3-yl)methanol;
(E)-N-isopropyl-3-(3-{6-[1-methyl-1-(methylsulfonyl)ethyl]-8-quinolinyl}phenyl)-2-[4-(methylsulfonyl)phenyl]-2-propenamide;
(E)-3-{3-[6-(1-cyano-1-methylethyl)-8-quinolinyl]phenyl}-2-[4-(methylsulfonyl)phenyl]-2-propenoic acid;
2-methyl-2-[8-(3-{(E)-2-(3-methyl-1,2,4-oxadiazol-5-yl)-2-[4-(methylsulfonyl)phenyl]ethenyl}-phenyl)-6-quinolinyl]propanenitrile;
(E)-3-{3-[6-(1-cyano-1-methylethyl)-8-quinolinyl]phenyl}-2-[4-(methylsulfonyl)phenyl]-2-propenamide;
(E)-N-(tert-butyl)-3-{3-[6-(1-cyano-1-methylethyl)-8-quinolinyl]phenyl}-2-[4-(methylsulfonyl)-phenyl]-2-propenamide;
(E)-3-[3-(6-isopropyl-8-quinolinyl)phenyl]-2-[4-(methylsulfonyl)phenyl]-2-propenoic acid;
6-isopropyl-8-(3-{(E)-2-(3-methyl-1,2,4-oxadiazol-5-yl)-2-[4-(methylsulfonyl)phenyl]-ethenyl}phenyl)quinoline;
(E)-3-(3-{6-[1-methyl-1-(methylsulfonyl)ethyl]-8-quinolinyl}phenyl)-2-[4-(methylsulfonyl)phenyl]-1-(1-pyrrolidinyl)-2-propen-1-one;
(E)-N-cyclopropyl-3-(3-{6-[1-methyl-1-(methylsulfonyl)ethyl]-8-quinolinyl}phenyl)-2-[4-(methylsulfonyl)phenyl]-2-propenamide;
(E)-N-(tert-butyl)-3-(3-{6-[1-methyl-1-(methylsulfonyl)ethyl]-8-quinolinyl}phenyl)-2-[4-(methylsulfonyl)phenyl]-2-propenamide;
8-{3-[2,2-bis(4-chlorophenyl)vinyl]phenyl}-6-isopropylquinoline;
6-isopropyl-8-(3-{(E/Z)-2-(6-methyl-3-pyridinyl)-2-[4-(methylsulfonyl)phenyl]ethenyl}-phenyl)quinoline;
6-isopropyl-8-(3-{(E/Z)-2-(5-methyl-2-pyridinyl)-2-[4(methylsulfonyl)phenyl]-ethenyl}phenyl)quinoline;
8-(3-{2,2-bis[4-(methylsulfonyl)phenyl]vinyl}phenyl)-6-isopropylquinoline;
2-methyl-2-[8-(3-{(E/Z)-2-(5-methyl-2-pyridinyl)-2-[4-(methylsulfonyl)phenyl]ethenyl}phenyl)-6-quinolinyl]propanenitrile;
2-[8-(3-{2,2-bis[4-(methylsulfonyl)phenyl]vinyl}phenyl)-6-quinolinyl]-2-methylpropanenitrile;
2-methyl-2-(8-{3-[(E)-2-[4-(methylsulfonyl)phenyl]-2-(2-pyridinyl)ethenyl]phenyl}-6-quinolinyl)propanenitrile;
6-[1-methyl-1-(methylsulfonyl)ethyl]-8-(3-{(E/Z)-2-(5-methyl-2-pyridinyl)-2-[4-(methylsulfonyl)phenyl]ethenyl}phenyl)quinoline;
2-(6-{(E)-2-(3-{6-[1-methyl-1-(methylsulfonyl)ethyl]-8-quinolinyl}phenyl)-1-[4-(methylsulfonyl)phenyl]ethenyl}-3-pyridinyl)-2-propanol;
(35 )-4-{2-[3,4-Bis(difluoromethoxy)phenyl]-2-{5-[2-(1-hydroxy-1-methyl)ethyl]-thiazolyl}ethyl}pyridine N-oxide;
chiral 4-{2-[3,4-Bis(difluoromethoxy)phenyl]-2-{5-[2-(1-hydroxy-1-methyl)ethyl]-thiazolyl}ethyl}pyridine N-oxide;
(±/±)-4-{2-[3,4-Bis(difluoromethoxy)phenyl]-2-[5-(2-(1-hydroxy-2,2,2-trifluoro)ethyl)-thiazolyl]ethyl}pyridine;
(±/±)-4-{2-[3,4-Bis(difluoromethoxy)phenyl]-2-[5-(2-(1-hydroxy-2,2,2-trifluoro)-ethyl)thiazolyl]ethyl}pyridine N-oxide;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine N-oxide;
(±/±)-4-({2-[3,4-Bis(difluoromethoxy)phenyl]-2-{5-[2-(1-hydroxy-1-trifluoromethyl)ethyl]-thiazolyl}ethyl}pyridine N-oxide;
(±/±)-4-{2-[3,4-Bis(difluoromethoxy)phenyl]-2-[5-(2-phenyhmethanol)thiazolyl]ethyl}pyridine N-oxide;
(±/±)-4-{2-[3,4-Bis(difluoromethoxy)phenyl]-2-[5-(2-(1-hydroxy-1-phenyl)ethyl)-thiazolyl]ethyl}pyridine N-oxide;
(±/±)-4-{2-[3,4-Bis(difluoromethoxy)phenyl]-2-[5-(2-(1-hydroxy-1-phenyl-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-phenyl)propyl)-thiazolyl]ethyl}pyridine N-oxide;
(±/±)-4-{2-[3,4-Bis(difluoromethoxy)phenyl]-2-[5-(2-cyclohexylmethanol)-thiazolyl]ethyl}pyridine;
(±/±)-4-{2-[3,4-Bis(difluoromethoxy)phenyl]-2-[5-(2-(1-hydroxy-1-cyclohexyl-2,2,2-trifluoromethyl)ethyl)thiazolyl]ethyl}pyridine N-oxide;
(±/±)-4-{2-[3,4-Bis(difluoromethoxy)phenyl]-2-[5-(2-(1-hydroxy-1-(4-ethyl)phenyl)ethyl)-thiazolyl]ethyl}pyridine N-oxide;
(±/±)-4-{2-[3,4-Bis(difluoromethoxy)phenyl]-2-[5-(2-(1-hydroxy-1-(4-ethyl)phenyl-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-(4-fluoro)phenyl)-ethyl)thiazolyl]ethyl}pyridine N-oxide;
(±/±)-4-{2-[3,4-Bis(difluoromethoxy)phenyl]-2-[5-(2-(1-hydroxy-1-(4-fluoro)phenyl-2,2,2-trifluoro)ethyl)thiazolyl]ethyl}pyridine N-oxide;
(±/±)-4-{2-[3,4-Bis(difluoromethoxy)phenyl]-2-[5-(2-(1-hydroxy-1-(5-bromopyridin-2-yl)-2,2,2-trifluoro)ethyl)thiazolyl]ethyl}pyridine N-oxide;
(±/±)-4-{2-[3,4-Bis(difluoromethoxy)phenyl]-2-[5-(2-(2-hydroxy-1-(6-bromopyridin-3-yl)-2,2,2-trifluoro)ethyl)thiazolyl]ethyl}pyridine N-oxide;
(±)-4-{2-[3,4-Bis(difluoromethoxy)phenyl]-2-{5-[2-(1-hydroxy)cyclobutyl]thiazolyl}-ethyl}pyridine N-oxide;
(±)-4-{2-[3,4-Bis(difluoromethoxy)phenyl]-2-{5-[2-(1-hydroxy)cyclohexyl]thiazolyl}-ethyl}pyridine N-oxide;
(±)-4-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 4-{2-[(3-cyclobutyloxy-4-difluoromethoxy)phenyl]-2-(5-[2-(1-hydroxy-1-methyl)ethyl]thiazolyl}ethyl}pyridine N-oxide;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
chiral 3-{2-[(3-cyclobutyloxy-4-difluoromethoxy)phenyl]-2-{5-[2-(1-hydroxy-1-methyl)ethyl]thiazolyl}ethyl}pyridine;
chiral 3-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 3-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 3-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
(±)-4-{2-[(3-cyclopropyloxy-4-difluoromethoxy)phenyl]-2-{5-[2-(1-hydroxy-1-methyl)ethyl]thiazolyl}ethyl}pyridine N-oxide;
(±)-3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
chiral 3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
or a pharmaceutically acceptable salt thereof.

22. Method of claim 17 wherein the PDE4 inhibitor is selected from:
2-(trans)-{3′-[3-[(cyclopropylamino)carbonyl]-4-oxo-1,8-naphthyridin-1(4H)-yl]-3-fluoro-1,1′-biphenyl-4-yl}cyclopropanecarboxylic acid;
2-(trans)-{3-chloro-3′-[3-[(cyclopropylamino)carbonyl)-4-oxo-1,8-naphthyridin-1(4H)-yl]-1,1′-biphenyl-4-yl}cyclopropanecarboxylic acid;
2-(cis)-{3′-[3-[(cyclopropylamino)carbonyl]-4-oxo-1,8-naphthyridin-1(4H)-yl]-3-fluoro-1,1′-biphenyl-4-yl}cyclopropanecarboxylic acid;
(+)-(trans)-2-{3-fluoro-3′-[4-oxo-3-{[(2,2,2-trifluoroethyl)amino]carbonyl}-1,8-naphthyridin-1(4H)-yl]biphenyl-4-yl}cyclopropanecarboxylic acid;
1-((3′-[3-[(cyclopropylamino)carbonyl]-4-oxo-1,8-naphthyridin-1(4H)-yl]biphenyl-4-yl}methyl)cyclobutanecarboxylic acid;
(trans)-2-{3′-[3-[(cyclopropylamino)carbonyl]4-oxo-1,8-naphthyridin-1(4H)-yl]biphenyl-4-yl}-2-methylcyclopropanecarboxylic acid;
3-methyl-3-{3′-[4-oxo-3-{[(2,2,2-trifluoroethyl)amino]carbonyl}-1,8-naphthyridin-1(4H)-yl]biphenyl-4-yl}butanoic acid;
3-{3′-[3-[(cyclopropylamino)carbonyl]-4-oxo-1,8-naphthyridin-1(4H)-yl]biphenyl-4-yl}-2,2-dimethylpropanoic acid;
4-{3′-[3-[(cyclopropylamino)carbonyl]-4-oxo-1,8-naphthyridin-1(4H)-yl]biphenyl-4-yl}-3,3-dimethylbutanoic acid;
1-({3′-[4-oxo-3-([(2,2,2-trifluoroethyl)amino]carbonyl}-1,8-naphthyridin-1(4R)-yl]biphenyl-4-yl}methyl)cyclobutanecarboxylic acid;
2,2-dimethyl-4-{3′-[4-oxo-3-{[(2,2,2-trifluoroethyl)amino]carbonyl}-1,8-naphthyridin-1(4H)-yl]biphenyl-4-yl}butanoic acid;
2,2-dimethyl-3-{3′-[4-oxo-3-{[(2,2,2-trifluoroethyl)amino]carbonyl}-1,8-naphthyridin-1(4H)-yl]biphenyl-4-yl}propanoic acid;
2-(trans)-{3′-[4-Oxo-3-{[(2,2,2-trifluoroethyl)amino]carbonyl}-1,8-naphthyridin-1(4H)-yl]-1,1′-biphenyl-4-yl}cyclopropanecarboxylic acid; and
(−)-(trans)-2-{3′-[4-oxo-3-{[(2,2,2-trifluoroethyl)amino]carbonyl}-1,8-naphthyridin-1(4H)-yl]biphenyl-4-yl}cyclopropanecarboxylic acid.
